(19) United States
(12) Patent Application Publication
(10) Pub. No.: US 2007/0123467 A1
(43) Pub. Date: May 31, 2007

(54) Amniotic-derived peptide and uses thereof

(76) Inventors: Vladimer Bakhutashvili, Tbilisi (GE); Jordan D. Haller, McMurray, PA (US); Ivane Bakhutashvili, Fox Point, WI (US); Alexander Bakhutashvili, Tibilisi (GE); Ferdinando Nicoletti, Sicily (IT); Michael Thiry, Nessonvaux (BE); Alain Poncin, Boncelles (BE)

Correspondence Address: LAW OFFICES OF ALBERT WAI-KIT CHAN, LLC, WORLD PLAZA, SUITE 604, 141-07 20TH AVENUE, WHITESTONE, NY, 11357, US

(21) Appl. No.: 11/434,525

(22) Filed: May 15, 2006

Related U.S. Application Data
(63) Continuation-in-part of application No. PCT/US04/37800, filed on Nov. 12, 2004.
(60) Provisional application No. 60/520,458, filed on Nov. 13, 2003.
(60) Provisional application No. 60/520,430, filed on Nov. 13, 2003.
(60) Provisional application No. 60/611,619, filed on Sep. 20, 2004.

(51) Int. Cl. A61K 38/10; C07K 7/06; C12P 21/06; C07H 21/04

(52) U.S. Cl. 514/16; 530/329; 435/69.1; 435/320.1; 435/325; 536/23.1

(57) ABSTRACT

The present invention relates to the method(s) of synthesis of and the therapeutic and cosmetic applications of biologically active peptides for improving the appearance of skin, for hastening wound healing and for treating and/or preventing the progression of various conditions, injuries and diseases, including but not limited to viral hepatitis B and C, herpes zoster ganglioneuritis, diabetic peripheral polyneuropathy, nephrotic syndrome, juvenile rheumatoid arthritis, rheumatoid arthritis, psoriatic arthritis, bronchial asthma, respiratory infection, breast cancer, epilepsy, psoriasis, atherosclerosis and other forms of vascular obstructions, myocardial infarction, HIV and SARS infection, brain cell malfunction due to ischemia and trauma, pathologic consequences of ischemia-reperfusion, rejection reaction following organ transplantation, chemical and drug intoxication including but not limited to anesthetic, alcohol and morphine, cancer, type 1 diabetes mellitus, multiple sclerosis, septic shock (Gram negative sepsis), Parkinson's disease, type 2 diabetes mellitus, Alzheimer's disease, amyotrophic lateral sclerosis, hyperthyroidism, Guillain-Barre syndrome, systematic lupus erythematosus, parasitic infections, especially leishmaniasis, and other collagen diseases, and diseases in which apoptosis occurs.

This is a Continuation-In-Part application of PCT/US2004/037800, filed Nov. 12, 2004, which claims benefit of U.S. Ser. Nos. 60/520,458, Filed Nov. 13, 2003, 60/520,430, Filed Nov. 13, 2003, and 60/611,619, Filed Sep. 20, 2004. The contents of these preceding applications are hereby incorporated in their entireties by reference into this application.

Throughout this application, references are made to various publications. Disclosures of these publications in their entireties are hereby incorporated by reference into this application to more fully describe the state of the art to which this invention pertains.

### BACKGROUND OF THE INVENTION

The references cited herein are not admitted to be prior art to the claimed invention.

The present invention relates to the method(s) of synthesis of and the therapeutic and cosmetic applications of biologically active peptides for improving the appearance of skin, for hastening wound healing and for treating and/or preventing the progression of various conditions, injuries and diseases, including but not limited to viral hepatitis B and C, herpes zoster ganglioneuritis, diabetic peripheral polyneuropathy, nephrotic syndrome, juvenile rheumatoid arthritis, rheumatoid arthritis, psoriatic arthritis, bronchial asthma, respiratory infection, breast cancer, epilepsy, psoriasis, atherosclerosis and other forms of vascular obstructions, myocardial infarction, HIV and SARS infection, brain cell malfunction due to ischemia and trauma, pathologic consequences of ischemia-reperfusion, rejection reaction following organ transplantation, chemical and drug intoxication including but not limited to anesthetic, alcohol and morphine, cancer, type 1 diabetes mellitus, multiple sclerosis, septic shock (Gram negative sepsis), Parkinson's disease, type 2 diabetes mellitus, Alzheimer's disease, amyotrophic lateral sclerosis, hyperthyroidism, Guillain-Barre syndrome, systematic lupus erythematosus and other collagen diseases, and diseases in which apoptosis occurs.

Apoptosis, or programmed cell death, is a principal mechanism by which organisms eliminate unwanted cells. The deregulation of apoptosis, either excessive apoptosis or the failure to undergo it, has been implicated in a number of diseases such as cancer, acute and chronic inflammatory disorders, auto-immune, immune and allergic disorders, ischemic diseases and/or certain neurodegenerative disorders.

An important regulator of apoptosis is the tumor necrosis factor receptors (See Chan et al. A Role for Tumor Necrosis Factor Receptor 2 (TNFR-2) and Receptor-interacting Protein (RIP) in Programmed Necrosis and Anti-Viral Responses. JBC Papers in Press. Oct. 7, 2004.):
Tumor Necrosis Factor (TNF) is a pleiotropic cytokine that mediates diverse biological responses ranging from inflammation to cell death. TNF exerts its biological functions mainly through binding to its two cell surface receptors, i.e., TNFR-1 and TNFR-2. Studies have shown that TNFR-2 may enhance TNFR-1 signaling under certain conditions. Signaling of the pre-assembled TNFR-1 results in the recruitment of the dead domain (DD)-containing TRADD adapter. Subsequent binding of TRAF2 or the protein serine/threonine kinase RIP is critical for TNF-induced Jnk kinase and NF-κB activation, respectively. In addition, binding of FADD and caspase-8 or caspase-10 to TRADD can initiate the caspase cascade, which results ultimately in cell death by apoptosis.

About 1978 Vladimir (Lado) Bakhutashvili initiated research to identify an inexpensive source of interferons (IF) using human placental tissues with amniotic and chorionic membranes. The terms that have been used to describe the materials include “placental interferon”, “Plaferon” and “PL”.

This pharmacologically active agent was shown to contain the following IF fractions: alpha 85-90%, beta 8-10% and gamma 3-5%. Plaferon has been tested according to IF titer in Inter-national Units (IU) and is registered as an antiviral and immunomodulatory drug by the Georgian Ministry of Health Care.

Experimental evidence showed that Plaferon possessed additional properties that were unknown in interferons. A new pharmaceutical and therapeutic preparation was then manufactured from human amniotic membranes. This product was commercialized under the name Plaferon-LB (“PLB”). It contained no interferons yet it still possessed some properties that had been observed in Plaferon such as anti-hypoxic, anti-allergic, anti-toxic, immuno-modulative, and apoptosis-modulative. Plaferon-LB also is free of HIV, hepatitis B and C viruses and prions.

The production of Plaferon ceased in 1992 and the method of producing Plaferon was never publicly disclosed prior to the filing of U.S. patent application Ser. No. 09/928,178 and International PCT Application No. PCT/US01/41666. In addition, many of the active ingredients in Plaferon were also never disclosed.

Plaferon-LB was approved in 1992 by the government of the Republic of Georgia as a pharmaceutical with anti-allergic antiviral and immunomodulatory actions (Republic of Georgia, Ministry of Health, Registration Number A-0001). The method of manufacture of Plaferon-LB was disclosed in U.S. patent application Ser. No. 09/928,178, filed Aug. 9, 2001, and Patent Cooperation Treaty (PCT) Application Number, PCT/US01/41666, filed Aug. 9, 2001 with International Publication Number WO 02/12444, the contents of which are herein incorporated by reference in its entirety for all purposes. At the time of the filing of U.S. patent application Ser. No. 09/928,178 and International PCT Application No. PCT/US01/41666, neither the active ingredients of PLB nor the methods for isolating the biologically active constituents of Plaferon-LB, which the subject of this patent application, had been disclosed.

Experiments disclosed herein suggested that many biological activity of Plaferon and Plaferon-LB are carried by a small molecular weight peptide.

### SUMMARY OF THE INVENTION

In accordance with these and other objects of the invention; a brief summary of the present invention is presented. Some simplifications and omission may be made in the following summary, which is intended to highlight and introduce some aspects of the present invention, but not to limit its scope. Detailed descriptions of a preferred exemplary embodiment adequate to allow those of ordinary skill in the art to make and use the invention concepts will follow in later sections.

The present invention features a bioactive peptide originally found in PLB and now synthesized by methods as described herein, including synthesis by DNA recombinant technology, chemical synthesis, rDNA technology, chemical engineering, and/or polynucleotides encoding. The bioactive peptide originally found in PLB can also be obtained from animal amniotic membranes. The biologically active peptide also referred to herein as “LAJOR ACTIVE PEPTIDE” or “LAP”.In one embodiment, the LAP is having one of the amino acid sequences of SEQ ID NOs. 1-5.

In one aspect, this invention provides methods for improving the appearance of skin and hastening wound healing using a cosmetic, pharmaceutical and/or therapeutic composition containing LAP.

In another aspect, this invention provides a method for normalizing the biochemical parameters of liver function and immunologic indices in viral hepatitis patients using pharmaceutical and therapeutic compositions containing LAP.

In another aspect, this invention provides a method for immuno-modulation, normalizing the levels of the tumor serum marker, CA15.3, and increasing tumor-infiltrating CD5′ T-cells and CD11 macrophages in a breast cancer subject using pharmaceutical and therapeutic compositions containing LAP.

In a further aspect, this invention provides methods for treating and/or preventing the progression of various conditions, injuries and diseases including but not limited to herpes zoster ganglioneuritis, diabetic peripheral polyneuropathy, nephrotic syndrome, Idiopathic Nephropathy Syndrome, juvenile rheumatoid arthritis, rheumatoid arthritis, psoriatic arthritis, bronchial asthma, respiratory infection, breast cancer, epilepsy, psoriasis, atherosclerosis and other forms of vascular obstructions, myocardial infarction, HIV and SARS infection, brain cell malfunction due to ischemia and trauma of many organs, especially the heart and kidney, pathologic consequences of ischemia-reperfusion, rejection reaction following organ transplantation, chemical and anesthetic intoxications including but not limited to anesthetic, alcohol and morphine, cancer, type 1 diabetes mellitus, multiple sclerosis, septic shock (Gram negative sepsis), Parkinson's disease, type 2 diabetes mellitus, Alzheimer's disease, amyotrophic lateral sclerosis, hyperthyroidism, Guillain-Barre syndrome, parasitic infections, especially leishmanaisis, systematic lupus erythematosus and other collagen diseases, and ulcerative colitis.

In one embodiment, the peptides and their functional equivalents described herein can be used in the prevention and/or treatment of epilepsy. It has been shown that simultaneous use of anticonvulants of carbamasepin and PLB resulted in ceasing or decreased attacks in epilepsy patients (6). As shown below, results presented herein demonstrated that the peptides and their functional equivalents described herein possess the same functional properties as that of PLB. Hence, the peptides and their functional equivalents described herein can be formulated into medication capable of preventing or treating epilepsy.

In yet another aspect, this invention provides a method for treating diseases in which apoptosis occurs.

### DETAILED DESCRIPTION OF THE FIGURES

For the purposes of illustrating the invention, there is shown in the drawings forms which are presently preferred. It is to be understood however, that the present invention is not limited to the precise arrangements and instrumentalities depicted in the drawings.

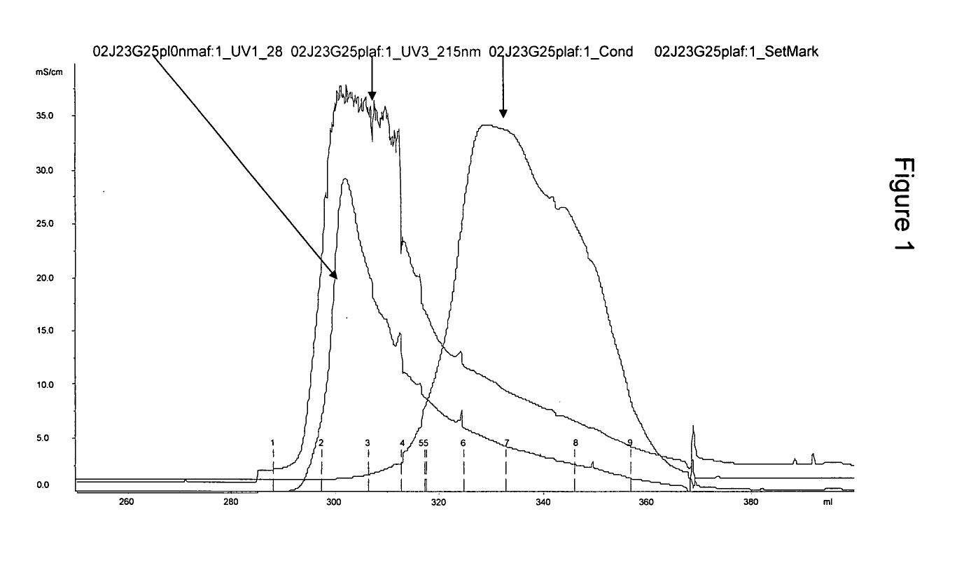

FIG. 1 shows the chromatographic profile of the purification of Plaferon-LB on Sephadex G25.

The FIG. 1 illustrates the chromatographic profile obtained after separation of the Plaferon compounds using Sephadex G25. The fractions containing the high molecular weight (>5000 Da) compounds and the fractions containing the low molecular weight (<5000 Da) compounds were pooled and freeze dried.

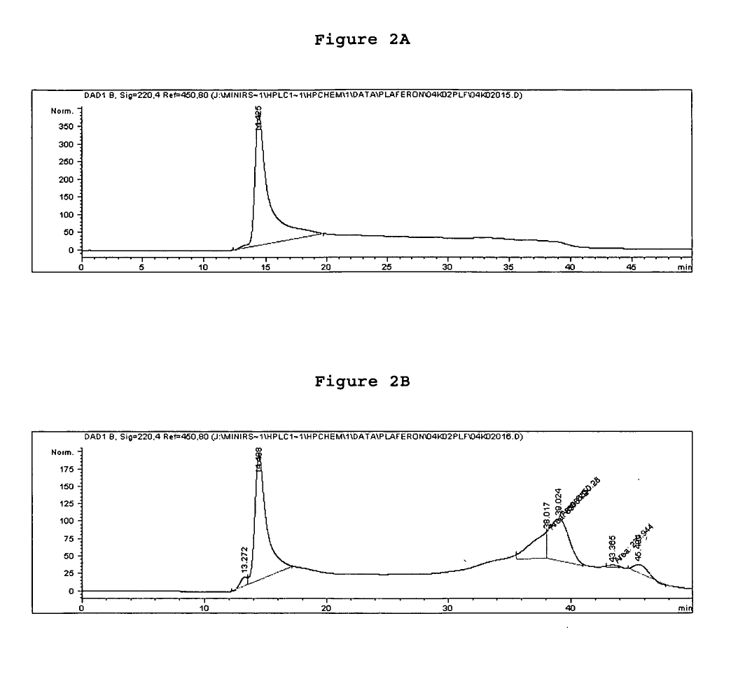

FIG. 2 shows the SE-HPLC of Plaferon-LB low and high molecular weight compounds on Superdex 30 HR 10/30.

FIG. 2(A) and 2(B) show that the pool containing the high molecular weight compounds contained only high molecular weight products (one peak in the exclusion volume, retention time=14.425 min) while the pool containing the low molecular weight compounds contained both a high molecular weight product (retention time: 14.408 min) and smaller peptides (retention time higher than 38 minutes).

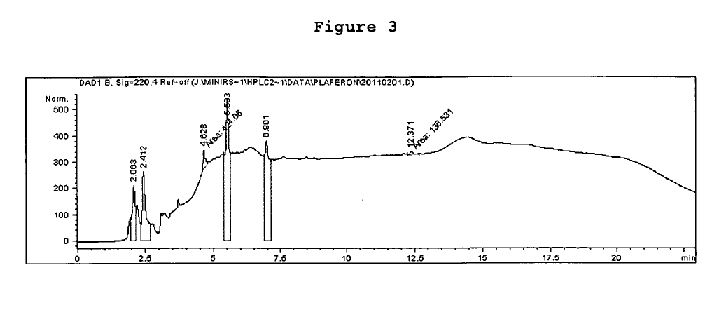

FIG. 3 shows the RP-HPLC of Plaferon-LB low molecular weight compounds.

RP-HPLC analysis detected several peptides in the Plaferon-LB low molecular weight compounds.

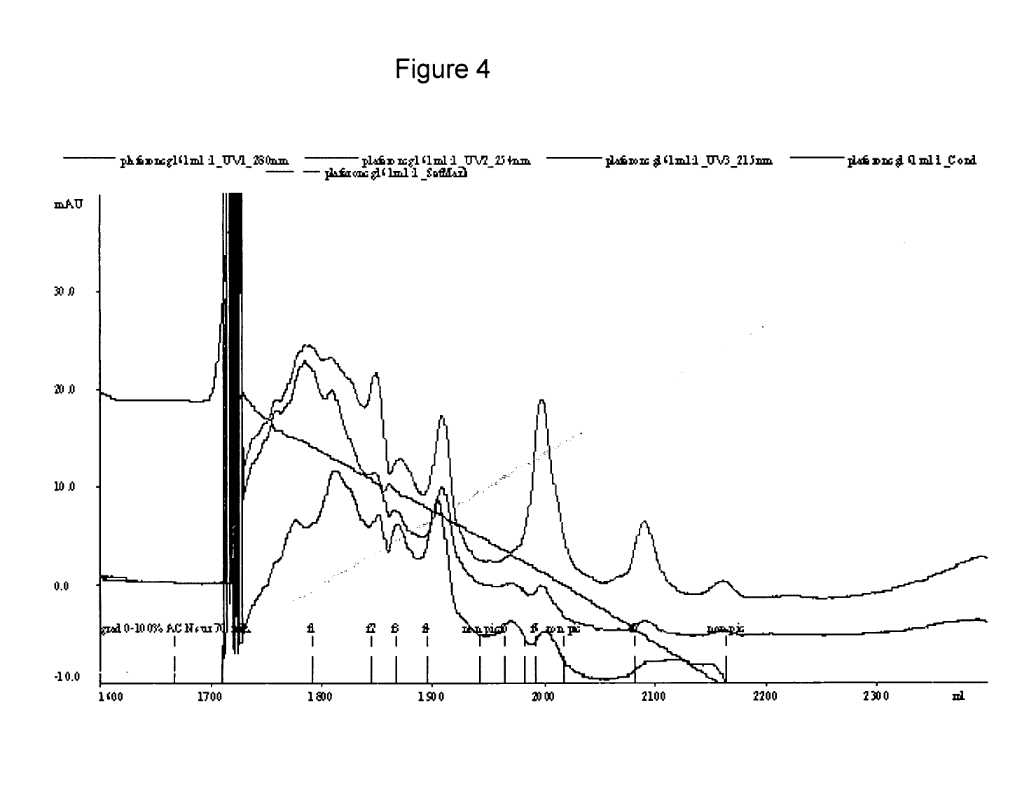

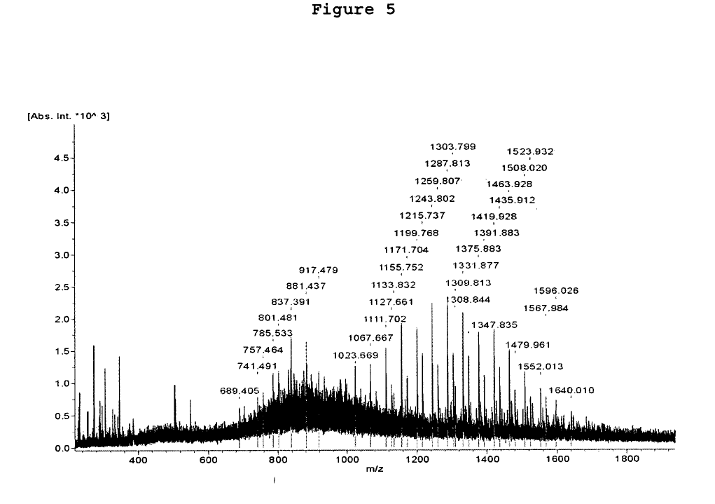

FIG. 4 shows the RP chromatography of low molecular weight compound of Plaferon-LB.

Chromatographic profile obtained using reverse chromatography (RP Chromatography) confirmed the analytical results obtained by RP-HPLC and eight (8) peaks were collected. The fractions that contain no peak were collected and pooled (fraction “RP Non pic”).

FIG. 5 shows the mass spectrometry of peptide in Fraction 4.

The sequence of the peptide in Fraction 4 was determined using mass spectrometry and NH₂ amino acid sequencing.

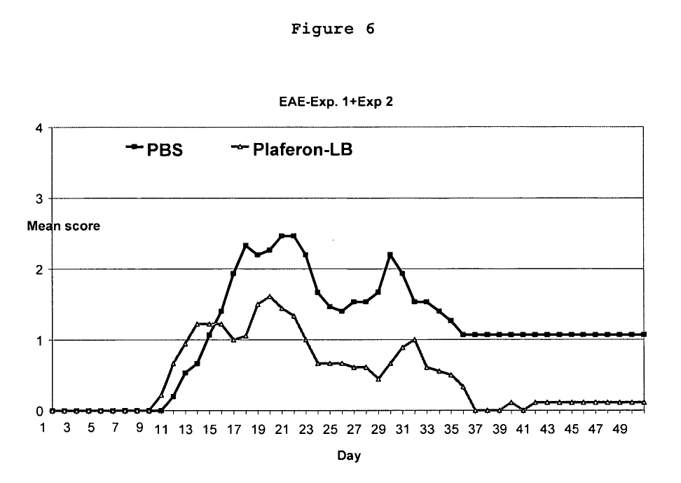

FIG. 6 shows the effects of PLB prophylaxis on the course of PR-EAE in DA rats

Table 6 is a comparison of the cumulative incidence of EAE among PLB-treated rats. Although the cumulative incidence of EAE among PLB-treated rats was not significantly different from that of control rats, relative to these latter animals, those treated with Plaferon-LB exhibited a milder course of the disease entailing lower EAE cumulative score and subsequent relapses of shorter duration and reduced severity.

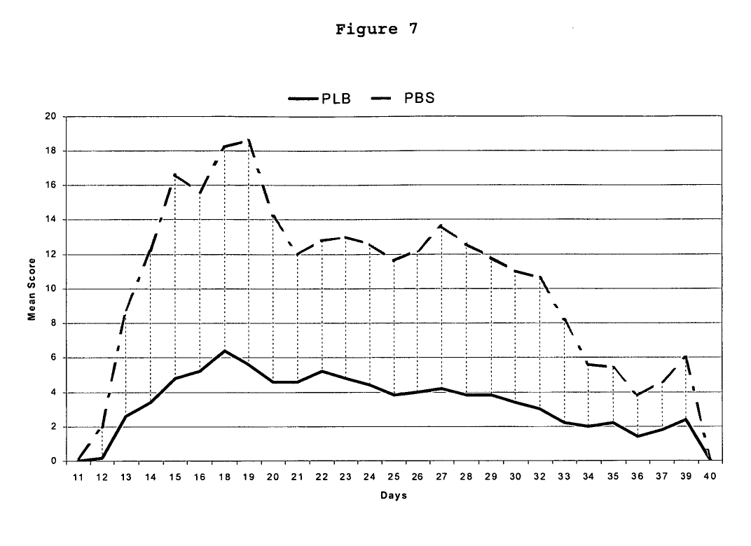

FIG. 7 shows the Plaferon-LB prophylaxis prevents OIA-induced arthritis in DA rats.

The course of OIA-arthritis was favorably influenced by PLB-prophylaxis. The treated rats exhibiting a markedly milder course of the disease that was mirrored by a significantly lower (p<0-0001) arthritic score than that recorded in control rats.

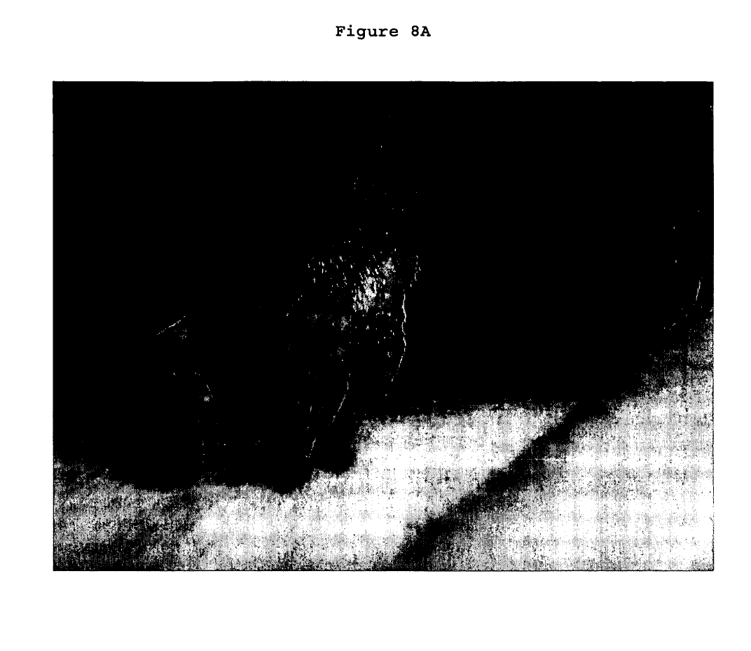

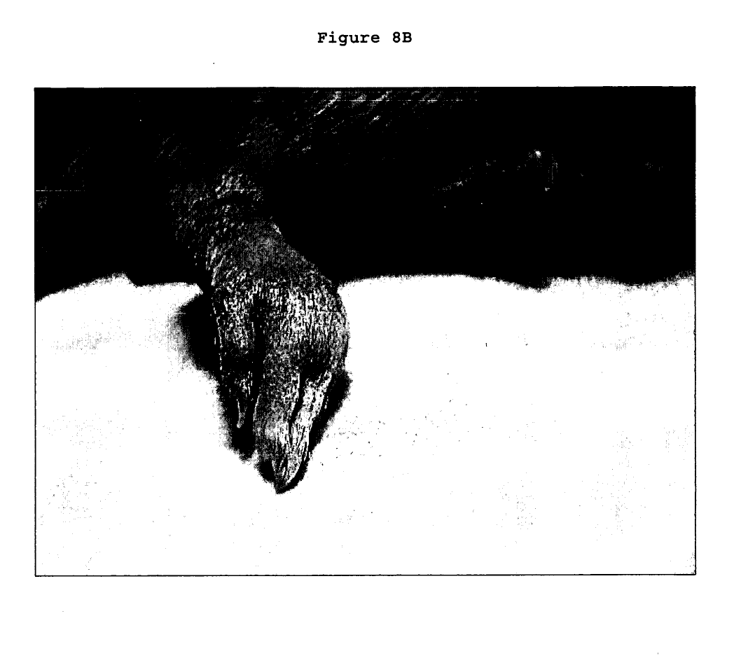

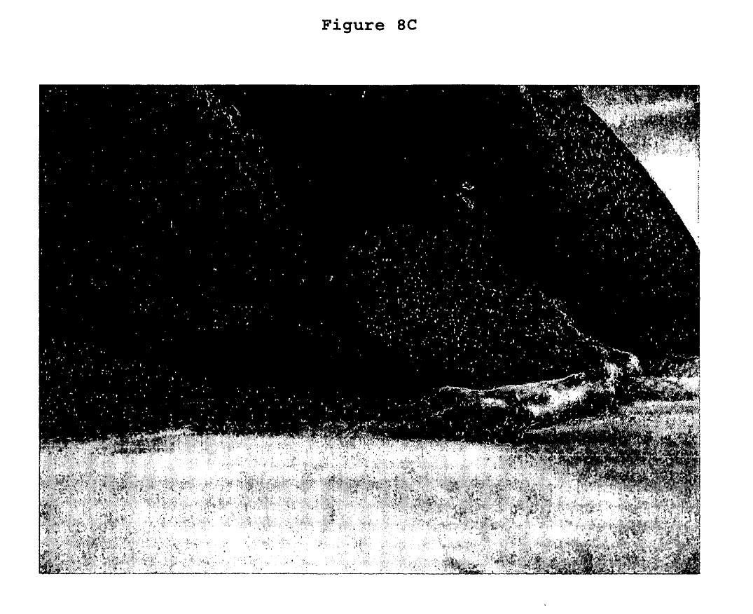

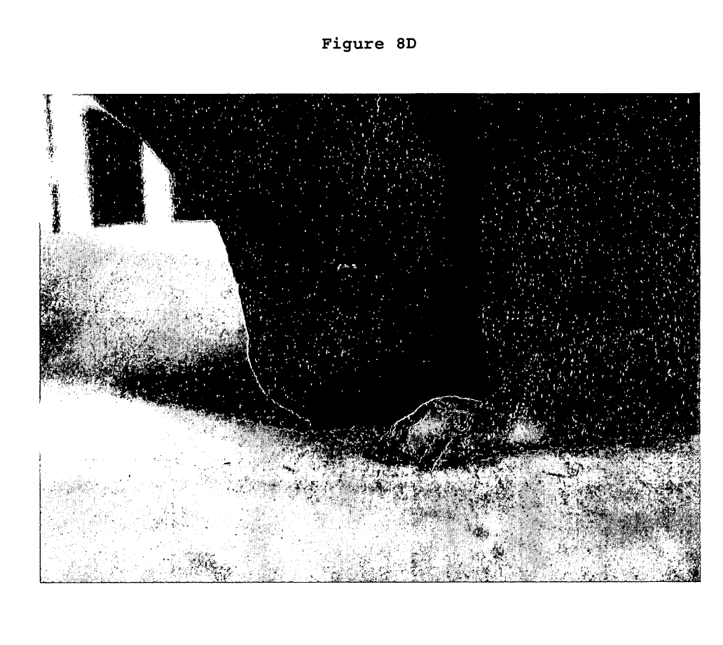

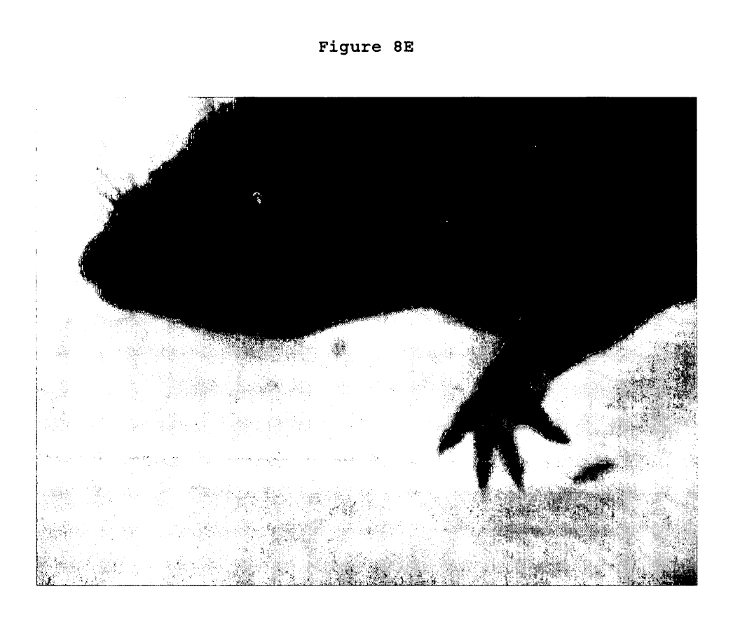

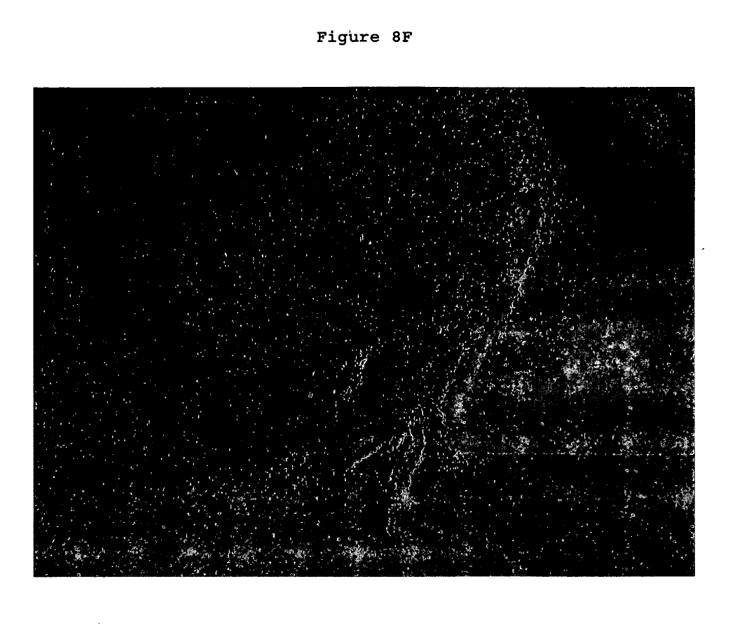

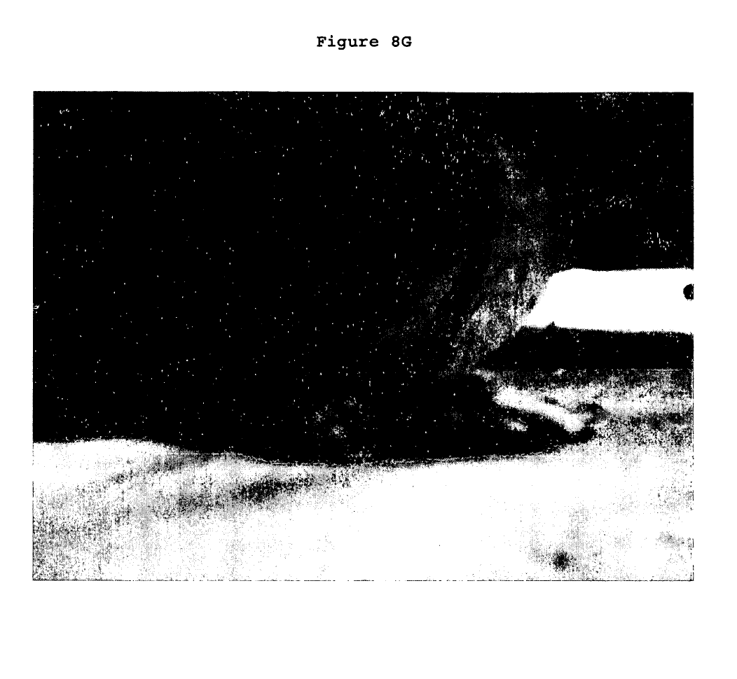

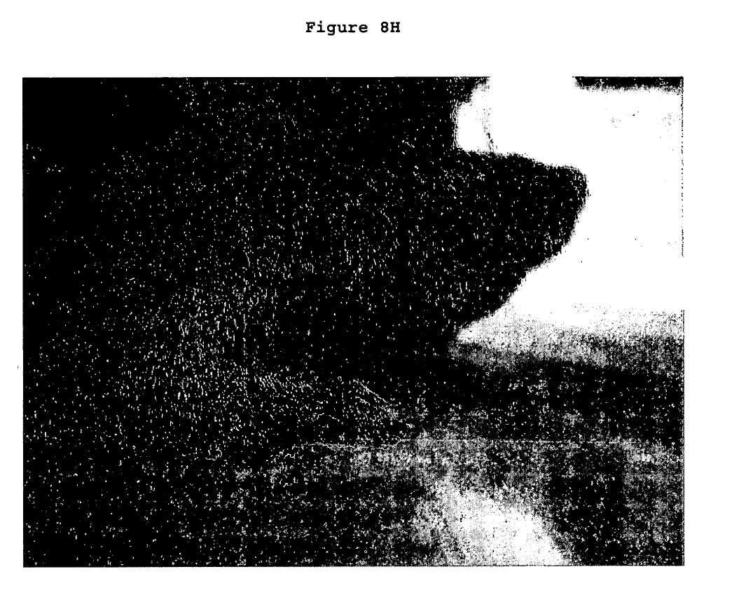

FIG. 8 shows the photographs taken from experiments using PLB Fraction 4 on oil-induced arthritis in DA rats.

FIGS. 8A-8D show oil-induced arthritis in control rats, and FIGS. 8E-H show rats treated with Fraction 4. Incidence of arthritis is 100% in control rats and 50% in Fraction 4-treated rats. In addition those two animals treated with Fraction 4 that have developed arthritis have much milder disease score.

FIG. 9 shows the RP-HPLC chromatographic profiles of two different batches of Plaferon-LB.

Blue: “first” Plaferon-LB batch

Red: “second” Plaferon-LB batch

LAP (Lajor Active Peptide) is indicated by a black arrow.

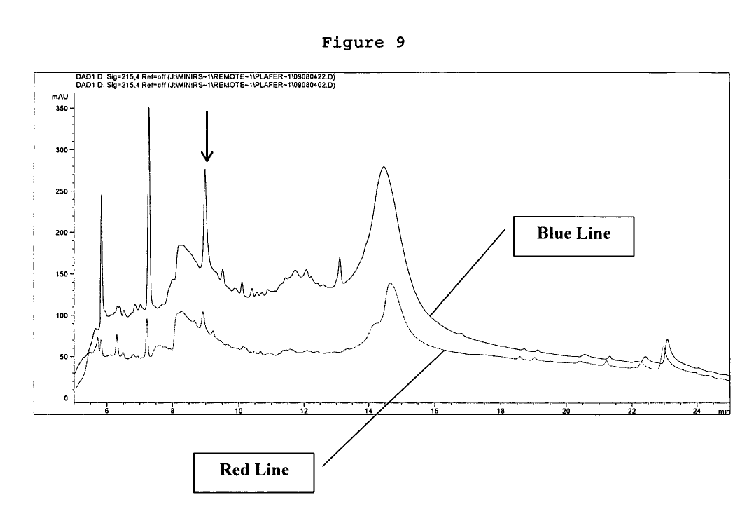

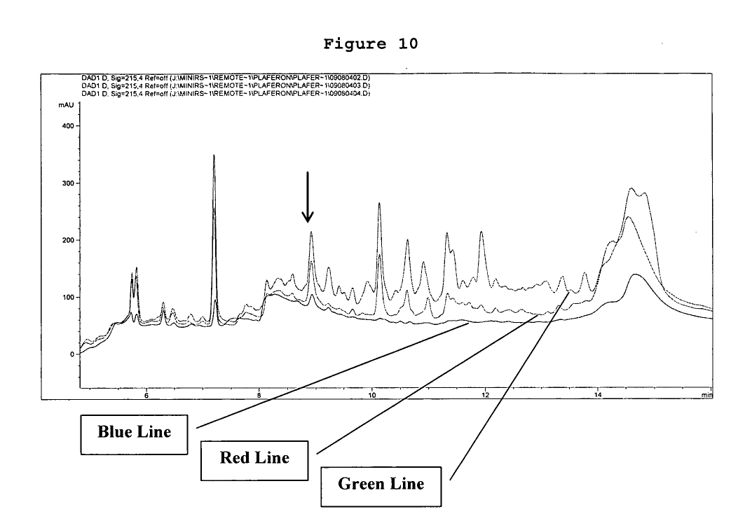

FIG. 10 shows the RP-HPLC chromatographic profiles of Plaferon-LB (final product and at two stage of manufacturing).

Blue: Plaferon-LB (final product).

Red: Plaferon-LB (stage I of manufacturing)

Green Plaferon-LB (stage II of manufacturing)

LAP (Lajor Active Peptide) is indicated by a black arrow.

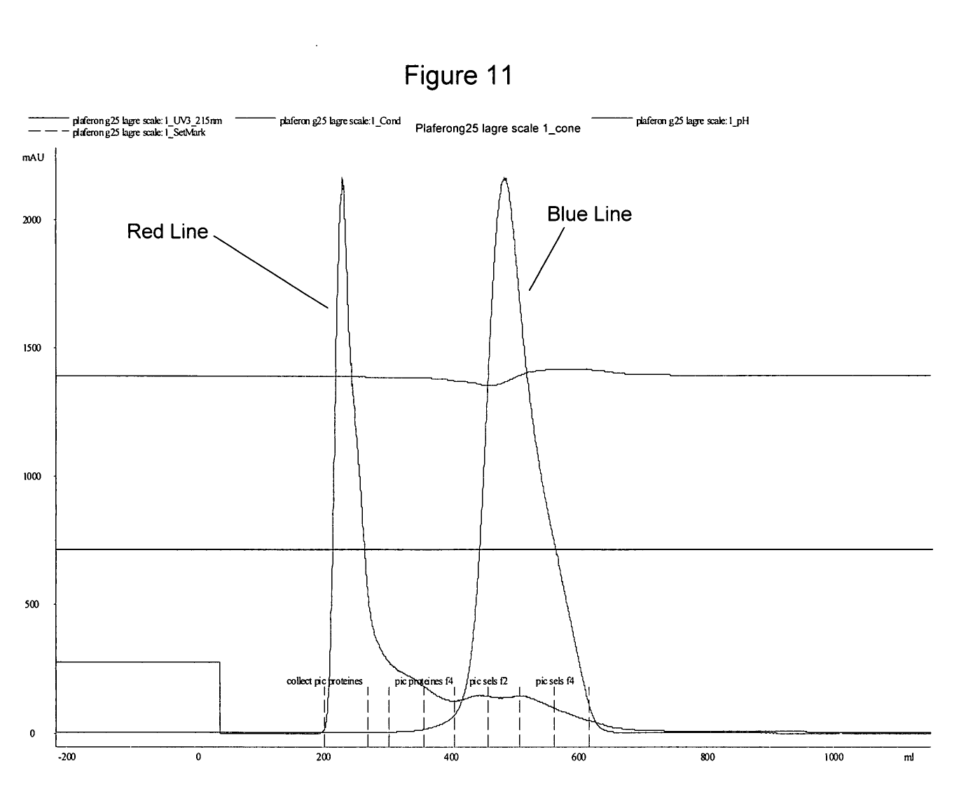

FIG. 11 shows the size exclusion chromatographic profile of Plaferon-LB.

FIG. 12 shows the RP-HPLC chromatographic profiles of Plaferon-LB (low and high MW after SE chromatography).

Blue Plaferon-LB (final product).

Red small MW fraction after SE chromatography

Green: High MW fraction after SE chromatography.

LAP (Lajor Active Peptide) is indicated by a black arrow.

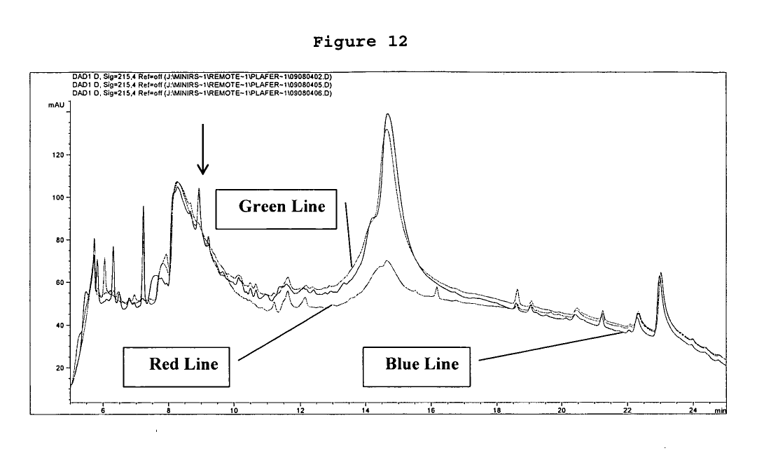

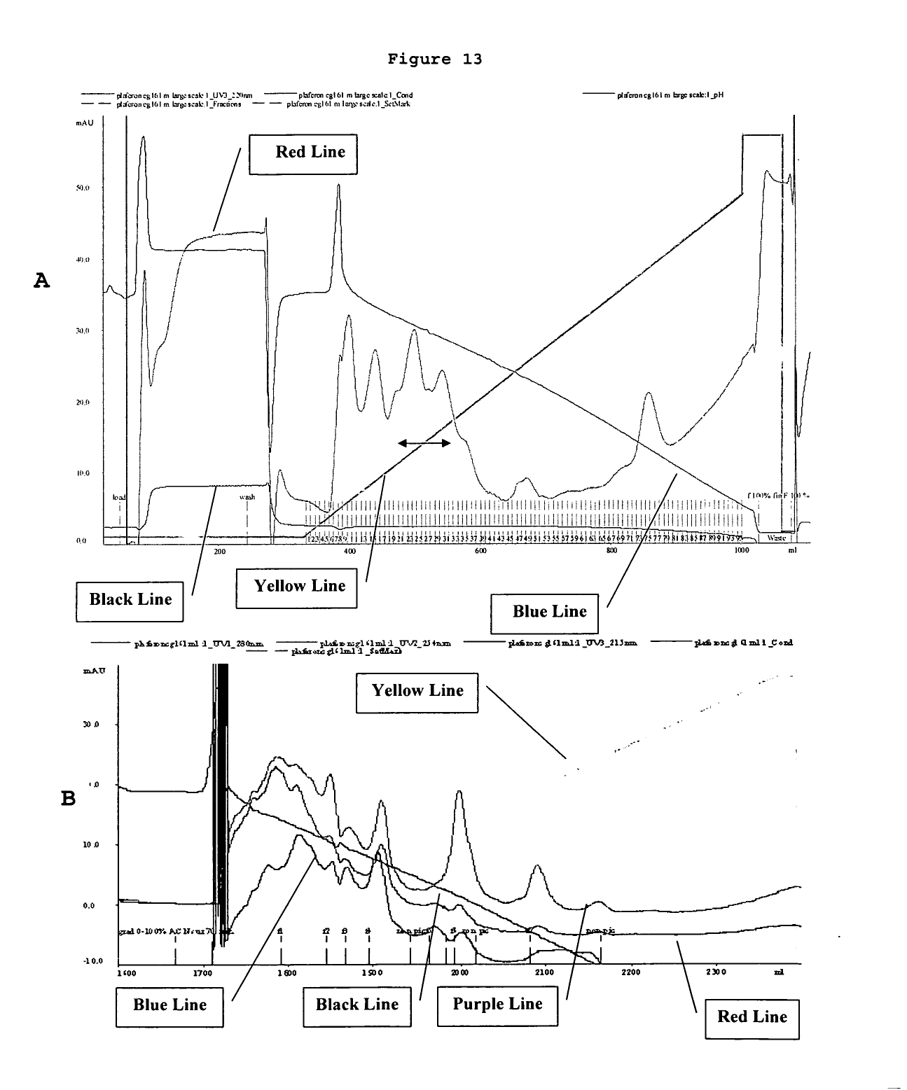

FIG. 13A-B shows the RP-HPLC chromatographic profiles of Plaferon-LB.

The double arrow shows the LAP in fraction 4 obtained from the first large scale purification.

FIG. 14 shows time and dose effects of LAP on LPS-induced lethality.

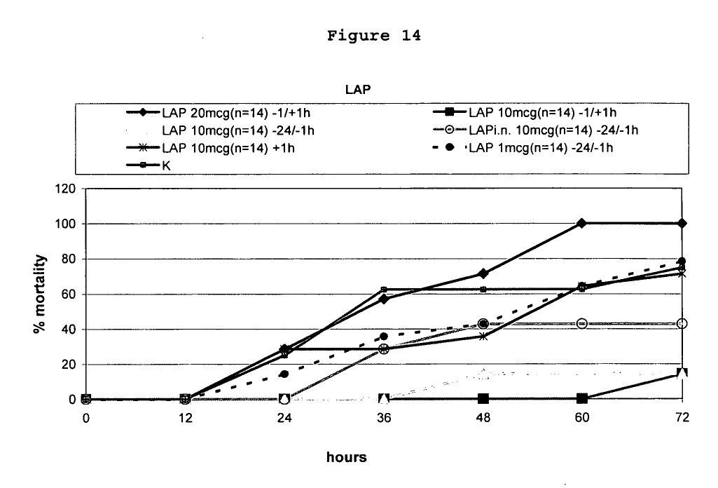

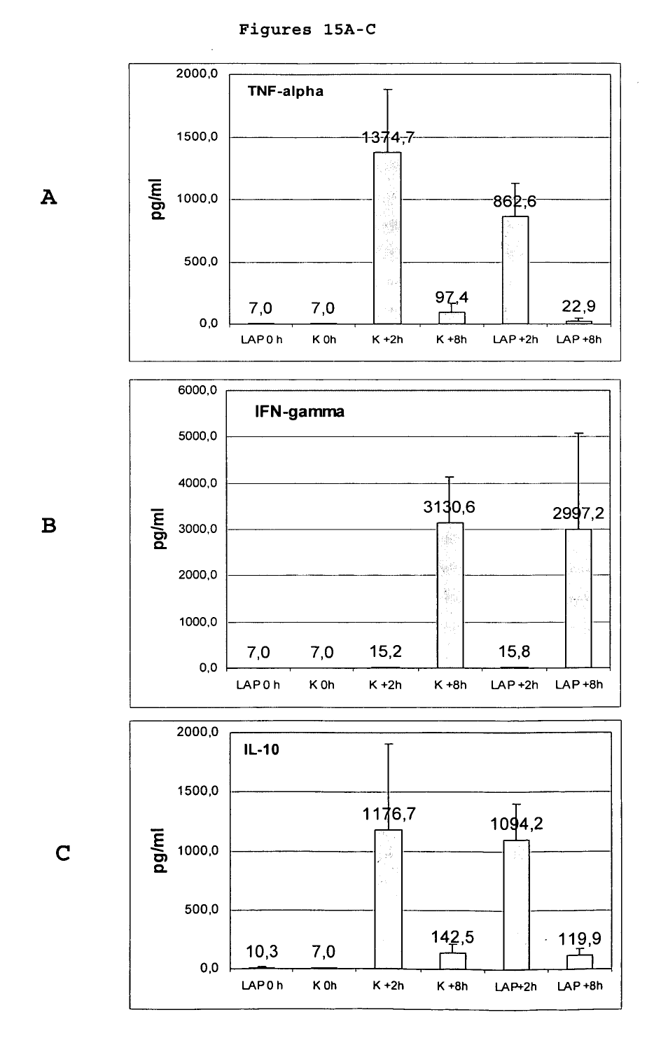

FIG. 15A-C shows LAP suppresses LPS-induced increase in circulating levels of TNF-α.

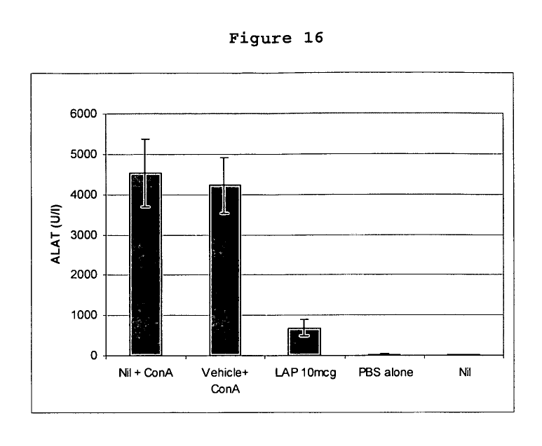

FIG. 16 shows reduction of Con A-induced ALAT increased by LAP prophylaxis.

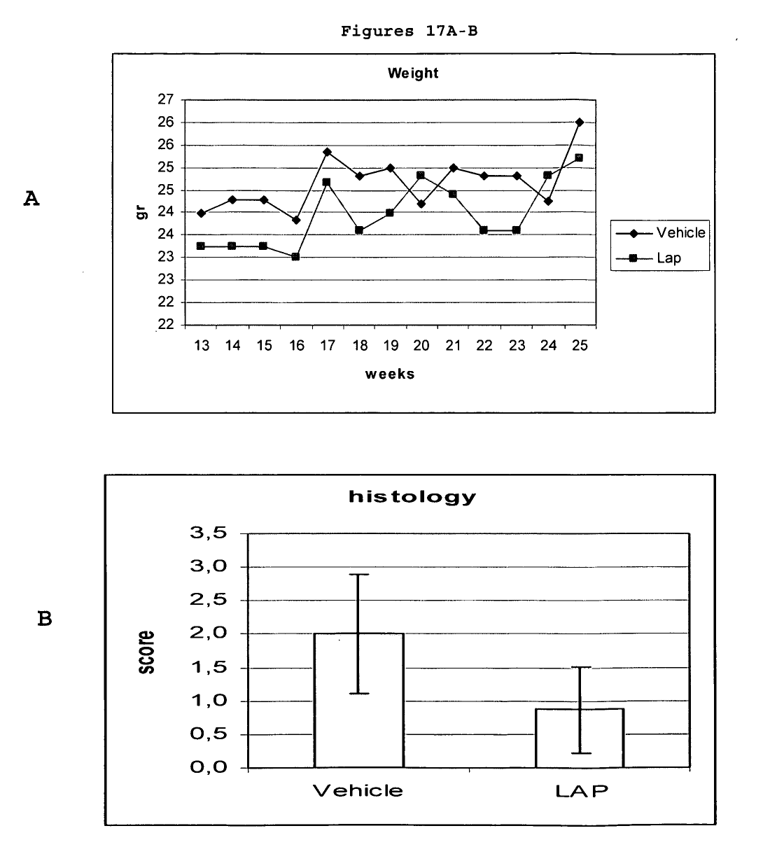

FIG. 17A shows the lack of effect of prolonged treatment (14-25 weeks) with LAP on body weight gain in NOD mice.

FIG. 17B shows the effects of early prophylactic treatment with LAP on the development of insulitis in NOD mice.

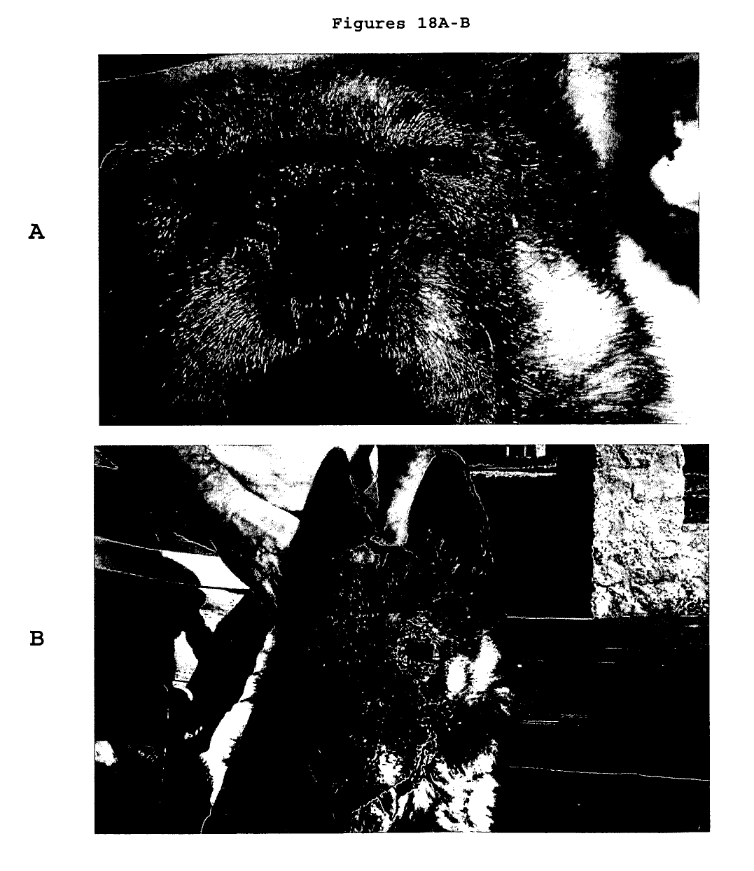

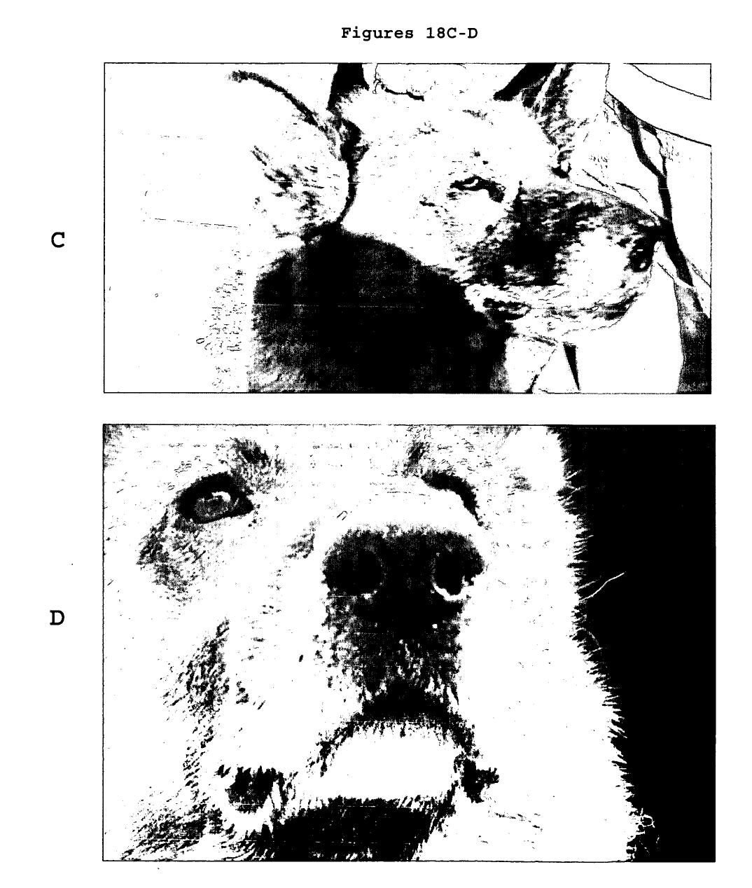

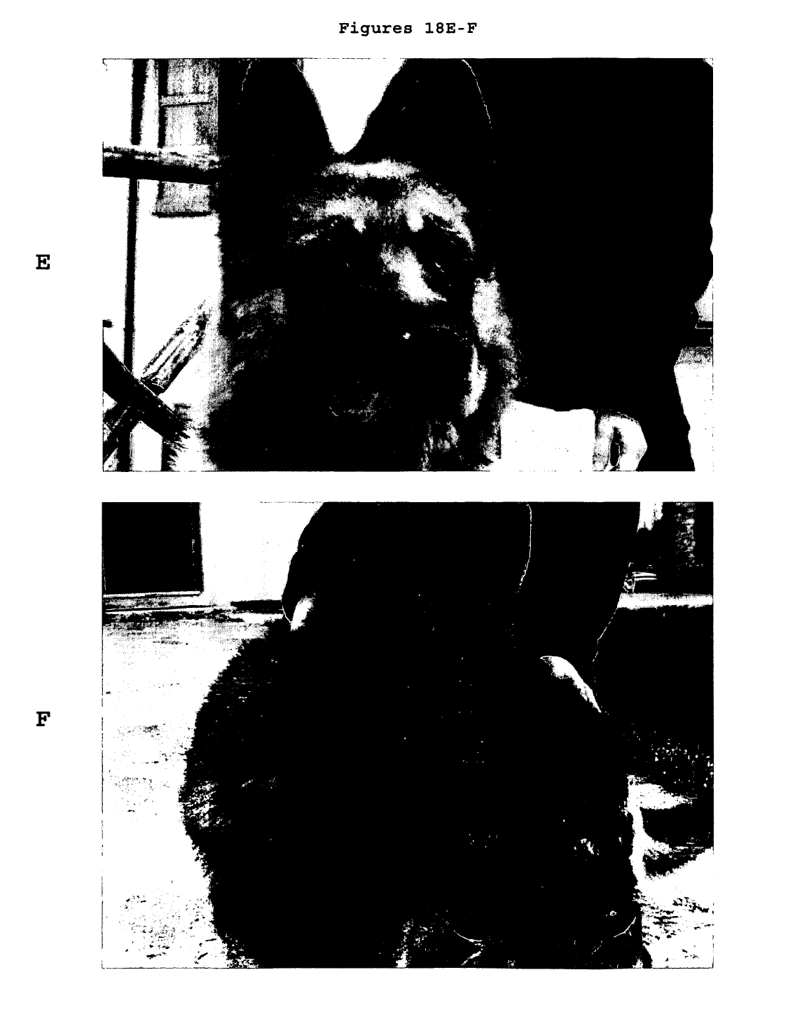

FIG. 18 shows the photographs taken from experiments using PLB in the treatment of leishmaniasis.

FIGS. 18A-C shows pictures of dogs with manifest clinical symptoms of leishmaniasis. FIGS. 18D-F shows pictures of dogs with substantial reduction of symptoms after administration of PLB.

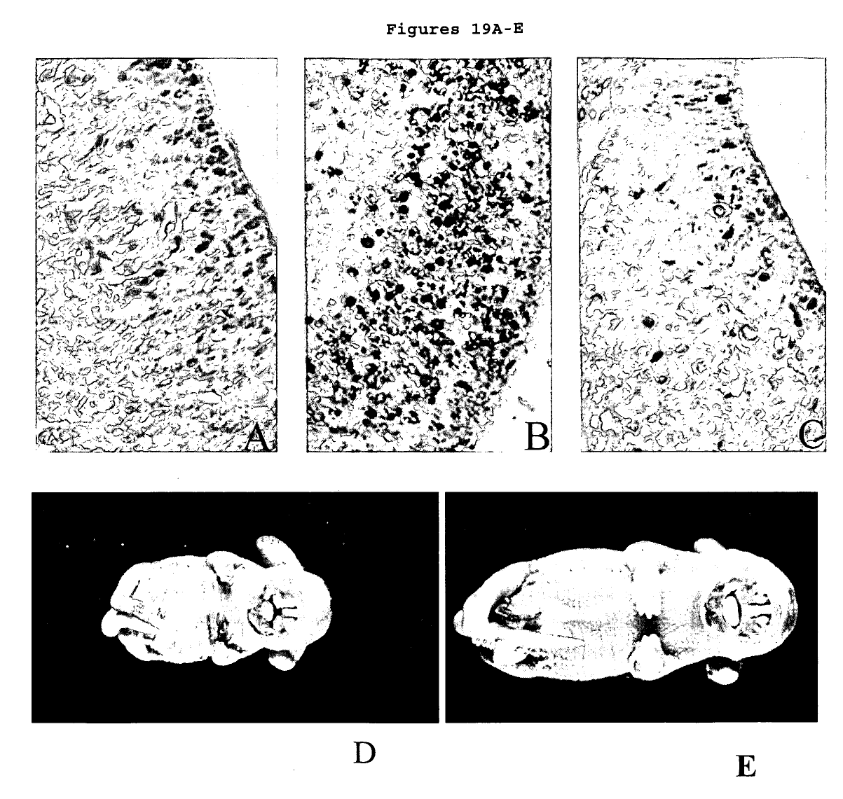

FIG. 19(A)-(C) shows sections (5-6 μM) of murine brain from all 3 groups of fetuses stained by TUNEL method. Dark spots represent apoptosis. (A) Control (no treatment). (B) CP only. (C) CP+Plaferon LB. FIG. 19(D) shows fetus from B group of animals (CP only) presented typical deformities, i.e., ectrodactily syndrome (anomaly of limbs), cleft pallet, kinked tail and low body mass. FIG. 19(E) shows shows fetus from C group of animals treated with CP and PLB with no deformity and normal weight/size.

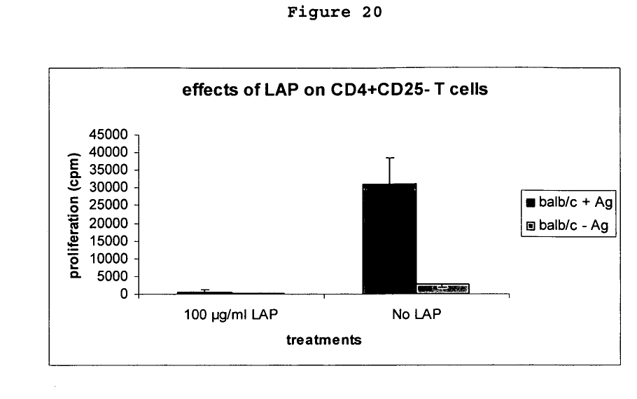

FIG. 20 shows the proliferation of CD4⁺CD25⁻ T cells in response to antigen-pulsed APCs in the presence or absence of LAP.

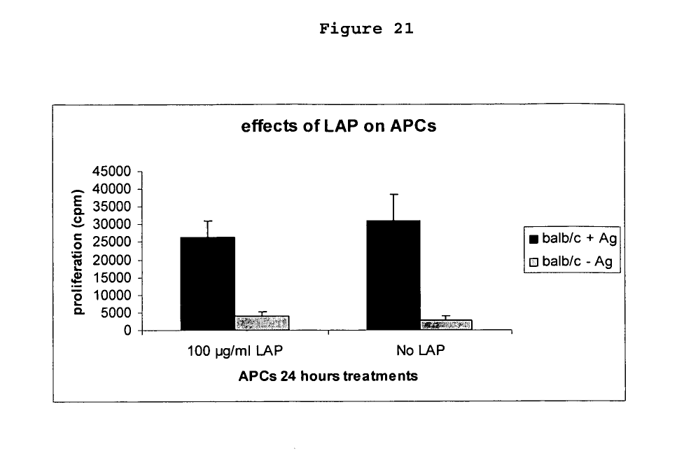

FIG. 21 shows the proliferation of CD4⁺CD25⁻ T cells in response to antigen-pulsed APCs pre-treated with or without LAP.

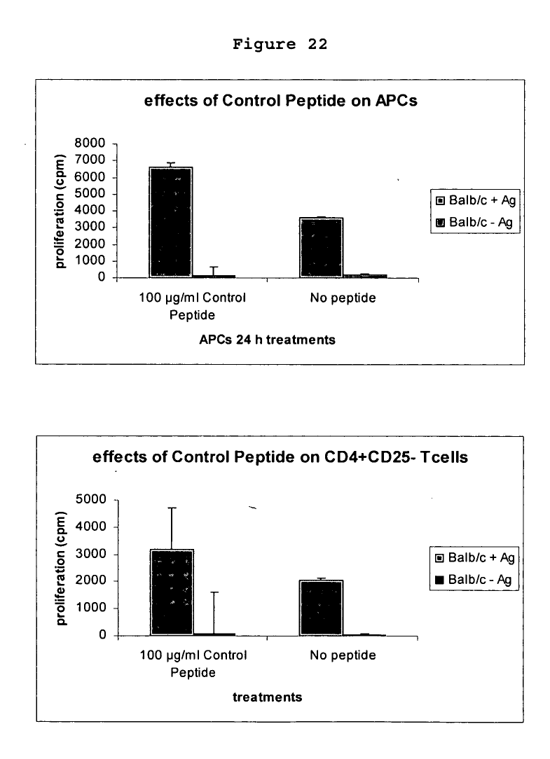

FIG. 22 shows the proliferation of CD4⁺CD25⁻ T cells in response to antigen-pulsed APCs in the presence of control peptide, as well as the proliferation of CD4⁺CD25⁻ T cells in response to antigen-pulsed APCs pre-treated with control peptide.

The present invention will be described in connection with a preferred embodiment, however, it will be understood that this is no intent to limit the invention to the embodiment described. On the contrary, the intent is to cover all alternatives, modifications, and equivalents as may be included within the spirit and scope of the invention as defined by the appended claims.

### DETAILED DESCRIPTION OF THE INVENTION

This invention provides an isolated peptide comprising amino acid sequence selected from the group consisting of SEQ ID NOs. 1-5 or its functional equivalents. In an embodiment, the serine in the sequence is modified to alter its activity state, localization, turnover, and/or interactions with other proteins. In another embodiment, the serine is modified by phosphorylation. The peptide with the phosphorylated serine has the amino acid sequence of NH2-NVSₚAVEIA-COOH (SEQ ID NO.2).

As used herein, protein modifications include but are not limited to altering the physical and chemical properties, folding, conformation distribution, stability, activity, and function of the proteins [103]. Modifications may involve changing the properties of a protein by proteolytic cleavage or by addition of a modifying group to one or more amino acids [101]. Moreover, the modification itself can act as an added functional group. Examples of the biological effects of protein modifications include phosphorylation for signal transduction, ubiquitination for proteolysis, attachment of fatty acids for membrane anchoring and association, glycosylation for protein half-life, targeting, cell:cell and cell:matrix interactions [103]. Other common types of protein modification include acetylation, methylation, fatty acid modification, Gylcosylphosphatidylinositol (GPI) anchor or membrane tethering of enzymes and receptors, hydroxyproline, sulfation, disulfide bond formation, deamidation, pyroglutamic acid, and ubiquitination [101].

Glycosylation has been known to have significant effects on protein folding, conformation distribution, stability and activity. Carbohydrates in the form of aspargine-linked (N-linked) or serine/threonine (O-linked) oligosaccharides are major structural components of many cell surface and secreted proteins [103]. Phosphorylation, principally on serine, threonine or tyrosine residues, is one of the most important and well-studied post-translational modifications. Phosphorylation plays critical roles in the regulation of many cellular processes including cell cycle, growth, apoptosis and signal transduction pathways [103].

Protein functions after modification can be determined using methods which are well known in the art, such as for example using sequence-based method that identifies and integrates relevant features that can be used to assign proteins of unknown function to functional classes [102].

As used herein, functional equivalents are compounds capable of performing equivalent functions as the above-described peptide. In one embodiment, proteins having the amino acid sequence of SEQ ID NOs. 1 or 2, allelic variants, species homologues and viral homologues thereof, as well as functional derivatives thereof including fragments which retain the biological characteristics of said amino acid sequence, and proteins that are substantially homologous thereto, which retain all characteristics of polypeptide of the invention.

A peptide is a molecule consisting of 2 or more amino acids. Peptides are smaller than proteins, which are also longer chains of amino acids. Molecules small enough to be synthesized from the constituent amino acids are, by convention, called peptides rather than proteins. The dividing line is about 25 to 50 amino acids.

Amino acids are the basic building block of proteins or polypeptides. They contain a basic amino (NH2) group, an acidic carboxyl (COOH) group and a side chain (R— of a number of different kinds) attached to an alpha carbon atom. The twenty (20) alpha amino acids have been recognized for their biological and pharmacological properties.

The twenty (20) biologically active alpha amino acids and their 3-letter and 1-letter abbreviations are: alanine-ala-A; arginine-arg-R; asparagine-asn-N; aspartic acid-asp-D; cysteine-cys-C; glutamine-gln-Q; glutamic acid-glu-E; glycine-gly-G; histidine-his-H; isoleucine-ile-I; leucine-leu-L; lysine-lys-K; methionine-met-M; phenylalanine-phe-F; proline-pro-P; serine-ser-S; threonine-thr-T; tryptophan-trp-W; tyrosine-tyr-Y; and valine-val-V.

These twenty alpha amino acids are classified into subgroups according to characteristics of the side chains:
Aliphatic-alanine, glycine, isoleucine, leucine, proline, valine
Aromatic-phenylalanine, tryptophan, tyrosine
Acidic-aspartic acid, glutamic acid
Basic-arginine, histidine, lysine
Hydroxylic-serine, threonine
Sulphur-containing-cysteine, methionine
Amidic (containing amide group)-asparagine, glutamine

This invention provides an isolated polypeptide comprising amino acid sequence selected from the group consisting of SEQ ID NOs. 1-5 or its functional equivalent. In an embodiment, the serine in the sequence is modified to alter its activity state, localization, turnover, and/or interactions with other proteins. In another embodiment, the serine is modified by phosphorylation.

It is the intention of this application to include modification of invention to include modification of these amino acids and the substitution of these amino acids.

A polypeptide is a compound consisting of a chain (10-100) of amino acids linked by peptide bonds.

This invention provides an isolated nucleic acid molecule encoding a polypeptide comprising an amino acid sequence selected from the group consisting of SEQ ID NOs. 1-5 or its functional equivalent. In one separate embodiment, this invention provides an isolated nucleic acid molecule encoding a peptide with sequence NH2-NVSAVEIA-COOH (SEQ ID NO.1) or NH2-NVSₚAVEIA-COOH (SEQ ID NO.2).

This invention provides an isolated peptide or polypeptide comprising amino acid sequence NVS or NVSP and its functional equivalents. In an embodiment, the amino acids after S do not suppress biological activity. In another embodiment, the serine is phosphorylated.

The present invention also provides a tripeptide having an amino acid sequence of NVS, NVSp, or a peptide comprising amino acid sequence X-N-(V or L)-blocking chemicals-Y, wherein the amino acids before N do not suppress biological activity and amino acids after V or L can be non-natural amino acids or other blocking chemicals such as phosphate or polyvinyl sulfone. In one embodiment, the tripeptide having an amino acid sequence of NVS or NVSp can be used to improve the skin condition of a subject.

The present disclosure also provides compositions comprising molecules or compounds that bind to or interact with LAP, including agonists or antagonists of LAP. Such agonists or antagonists of LAP include antibodies and antibody mimetics, as well as other molecules that can readily be identified by routine assays and experiments well known in the art.

Polyclonal antibodies are heterogeneous populations of antibody molecules derived from the sera of animals immunized with an antigen, such as LAP, or an antigenic functional derivative thereof. For the production of polyclonal antibodies, host animals may be immunized by injection with LAP supplemented with adjuvants according to procedures well known in the art.

Monoclonal antibodies, which are homogeneous populations of antibodies to a particular antigen, may be obtained by any technique that provides for the production of antibody molecules by continuous cell lines in culture. These include, but are not limited to the hybridoma technique (Kohler and Milstein, Nature 256:495-7 (1975), and U.S. Pat. No. 4,376,110); the human B-cell hybridoma technique (Kosbor et al., Immunology Today 4:72 (1983); Cote et al., Proc. Natl. Acad. Sci. USA, 80:2026-30 (1983)), and the EBV-hybridoma technique (Cole et al., in Monoclonal Antibodies And Cancer Therapy, Alan R. Liss, Inc., New York, pp. 77-96 (1985)). Such antibodies may be of any immunoglobulin class including IgG, IgM, IgE, IgA, IgD and any subclass thereof.

In addition, techniques developed for the production of “chimeric antibodies” (Morrison et al., Proc. Natl. Acad. Sci. 81:6851-6855 (1984); Takeda et al., Nature 314:452-54 (1985)) by splicing the genes from a mouse antibody molecule of appropriate antigen specificity together with genes from a human antibody molecule of appropriate biological activity can be used.

Alternatively, techniques described for the production of single chain antibodies (U.S. Pat. No. 4,946,778; Bird, Science 242:423-26 (1988); Huston et al., Proc. Natl. Acad. Sci. USA 85:5879-83 (1988); and Ward et al., Nature 334:544-46 (1989)) can be adapted to produce gene-single chain antibodies. Antibody fragments that recognize specific epitopes may also be generated by known techniques. For example, such fragments include but are not limited to: the F(ab′)₂ fragments that can be produced by pepsin digestion of the antibody molecule and the Fab fragments that can be generated by reducing the disulfide bridges of the F(ab′)₂ fragments. Alternatively, Fab expression libraries may be constructed (Huse et al., Science 246:1275-81 (1989)) to allow rapid and easy identification of monoclonal Fab fragments with the desired specificity.

This invention provides an isolated nucleic acid molecule encoding a polypeptide which includes the amino acid sequence NVSₚ. In a separate embodiment, this invention provides an isolated nucleic acid molecule encoding a peptide with sequence NVSₚ.

As used herein, nucleic acid is defined as RNA or DNA encoding an isolated peptide or its functional equivalents or a polypeptide of the present invention, or is complementary to nucleic acids encoding such peptides or polypeptide.

This invention provides a vector comprising the nucleic acid molecule encoding an amino acid sequence selected from the group consisting of SEQ ID NOs. 1-5 or its functional equivalent.

As used herein, a vector is defined as any agent that acts as a carrier or transporter, as a virus or plasmid that conveys a genetically engineered DNA segment into a host cell.

This invention provides a cell containing the nucleic acid molecule or the vector of the nucleic acid molecule encoding the amino acid sequence selected from the group consisting of SEQ ID NOs. 1-5 or its functional equivalent.

This invention provides an expression system for the expression of the above-described polypeptide or peptide or its functional equivalents. In one embodiment, this invention provides an expression system comprising an isolated nucleic acid molecule or the vector of an isolated nucleic acid molecule encoding the amino acid sequence NH2-NVSAVEIA-COOH (SEQ ID NO.1).

This invention provides a method for producing an isolated peptide comprising amino acid sequence selected from the group consisting of SEQ ID NOs. 1-5 or its functional equivalent. The said isolated peptide or its functional equivalents or said isolated polypeptide are produced by introducing a nucleic acid molecule encoding said polypeptide into an appropriate cell and placing the cell in suitable conditions thereby permitting expression of the said peptide or its functional equivalents or said polypeptide.

In an embodiment, the above method further comprises recovery of said peptide and its functional equivalents or said polypeptide.

In a separate embodiment, the nucleic acid molecule is operatively linked to a regulatory element. Said regulator element include but are not limited to promoter, enhancer and motifs which are essential for gene expression.

In a further embodiment, the nucleic acid molecule is linked to a vector.

This invention provides a transgenic animal or chimera comprising the nucleic acid molecule or vector encoding the amino acid sequence selected from the group consisting of SEQ ID NOs. 1-5 or its functional equivalent. This invention also provides a method for producing the said transgenic animal or chimera.

In one embodiment, this invention provides an animal comprising the nucleic acid molecule encoding the amino acid sequence NH2-NVSAVEIA-COOH (SEQ ID NO.1) or the vector of nucleic acid molecule encoding the amino acid sequence NH2-NVSAVEIA-COOH (SEQ ID NO.1). This invention also provides a method for producing the said animal.

This invention provides a composition containing a suitable carrier and an isolated peptide comprising amino acid sequence selected from the group consisting of SEQ ID NOs. 1-5 or its functional equivalent.

As used herein, the term suitable carrier includes but is not limited to any suitable carrier for administering pharmaceutical compositions known to those of ordinary skill in the art. The type of carrier will vary depending on the mode of administration.

With regards to compositions for parenteral administration (e.g. subcutaneous injections), the term suitable carrier includes but is not limited to water, saline, alcohol, a fat, a wax or a buffer.

With regards to compositions for oral administration, the term suitable carrier includes but is not limited to any of the above carriers or a solid carrier, such as mannitol, lactose, starch, magnesium stearate, sodium saccharine, talcum, cellulose, glucose, sucrose, and magnesium carbonate, may be employed.

Biodegradable microspheres (e.g., polylactate polyglycolate) may also be employed as carriers for the pharmaceutical compositions of this invention.

This invention provides a pharmaceutical composition containing an effective amount of an isolated peptide comprising amino acid sequence selected from the group consisting of SEQ ID NOs. 1-5 or its functional equivalent in a pharmaceutically acceptable carrier.

As used herein, pharmaceutically acceptable carriers include but are not limited to any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCI., 15th Ed. (Mack Publ. Co., Easton (1975)). Pharmaceutically acceptable carriers could be selected from the group of a liquid, an aerosol, a capsule, a tablet, a pill, a powder, a gel, an ointment, a cream and a granule. In another embodiment, the pharmaceutically acceptable carrier comprises a controlled release formulation. In yet another embodiment, the pharmaceutically acceptable carrier is selected from the group of: water, phosphate buffered saline, Ringer's solution, dextrose solution, serum-containing solutions, Hank's solution, other aqueous physiologically balanced solutions, oils, esters, glycols, biocompatible polymers, polymeric matrices, capsules, microcapsules, microparticles, bolus preparations, osmotic pumps, diffusion devices, liposomes, lipospheres, cells, and cellular membranes.

In another embodiment, the above pharmaceutical composition may also contains an agent selected from the group consisting of antibiotics, wound healing agents, antioxidants, antivirals, antifungals, anti-ischemics, anti-injury, anti-aging, immunomodulatory, anti-hypoxic, anti-toxic, anti-allergic, antiwrinkle, anti-inflammatory anti-infectious, anti-immunogenic, anti-protozoal, anti-parasitic and anti-neoplastic [1, 2, 4, 7, 8, 10, 13, 15, 16, 18, 19, 20, 27, 28, 39, 50, 52, 60, 66].

In one embodiment, this invention provides a pharmaceutical composition containing an effective amount of an isolated peptide comprising amino acid sequence NH2-NVSAVEIA-COOH (SEQ ID NO.1) or its functional equivalents or an isolated polypeptide comprising amino acid sequence NH2-NVSAVEIA-COOH (SEQ ID NO.1) in a pharmaceutically acceptable carrier suitable for topical, sublingual, parenteral, or gastrointestinal administration or aerosolization.

In one embodiment, this invention provides a method for producing an isolated peptide comprising amino acid sequence selected from the group consisting of SEQ ID NOs. 1-5 or its functional equivalent by chemical synthesis or by genetic engineering.

This invention provides a method for protecting the retinal tissue of a subject by administering an effective amount of an isolated peptide comprising amino acid sequence NH2-NVSAVEIA-COOH (SEQ ID NO.1) or its functional equivalents or an isolated polypeptide comprising amino acid sequence NH2-NVSAVEIA-COOH (SEQ ID NO.1) to said subject [96]. In another embodiment, the peptides described herein or their functional equivalent can be formulated as eyedrops to inhibit eye surface capillaries growth and inflammations, e.g. eyedrops comprising the peptides described herein or their functional equivalent can provide therapeutic use in treating cornea burnt by caustic agent.

This invention provides a method for improving the skin appearance of a subject by contacting the skin surface of said subject with an effective amount of an isolated peptide comprising amino acid sequence selected from the group consisting of SEQ ID NOs. 1-5 or its functional equivalent.

As used herein, skin conditions include but are not limited to psoriasis, atopic dermatitis, herpes simplex, herpes zoster, eczemas, skin burns of different severity and origin, wrinkles, pigment spots. In an embodiment, the peptide or polypeptide of the present invention is mixed or coupled with a cosmetic carrier. As used herein, cosmetic carrier includes at least one additive ingredient such as agents, silicone oils, thickeners, perfume oils, turbidity-inducing agents, anionic surfactants, cationic surfactants, nonionic surfactants, amphoteric surfactants, moisturizing agents, dye stuffs, light-protective agents, antioxidants, luster-imparting agents and preservatives.

This invention provides a method for treating a hepatitis patient with an effective amount of the above-described peptide or its functional equivalents or the above-describe polypeptide. In an embodiment the above-described peptide or its functional equivalents or polypeptide can normalize the biochemical parameters of liver function and immunologic indices in an acute viral hepatitis B or hepatitis C subject, speed the recovery from symptoms of the disease, or prevent recurrence of the disease in a subject [14, 26, 32, 41, 42, 53, 97].

This invention provides a method for treating a herpes zoster ganglioneuritis subject with an effective amount of the above-describe peptide or its functional equivalents or polypeptide. In an embodiment, the said peptide and/or its functional equivalents or polypeptide can normalize cell counts of CD3+, CD4+, CD8+, and T-cells carrying HLA-DR antigens and improve neurological symptoms in a herpes zoster ganglioneuritis subject [45].

This invention provides a method for normalizing levels of CD3+ and CD4+ T-cell phenotypes in a diabetic peripheral polyneuropathy subject.

This invention provides a method for treating a patient with nephrotic syndrome by administering to the subjet an effective amount of the above-described peptide or its functional equivalent [29]. In one embodiment, this invention provides a method for treating or preventing progression of nephrotic syndrome in a child-patient comprising administering an effective amount of an isolated peptide comprising amino acid sequence NH2-NVSAVEIA-COOH (SEQ ID NO.1) or its functional equivalents or an isolated polypeptide comprising amino acid sequence NH2-NVSAVEIA-COOH (SEQ ID NO.1) to the subject [29].

The above-described peptide or its functional equivalents or the above-described polypeptide are capable of promoting earlier and prolonged clinical laboratory remission in a child-patient with Idiopathic Nephropathy Syndrome (INS) and correcting the reduction in CD3+ and CDB+ T lymphocytes [29].

This invention provides a method for treating or preventing progression of arthritis in a subject comprising administering an effective amount of the above-described peptide or its functional equivalents or the above-described polypeptide. In an embodiment, the said peptide or its functional equivalents or the said polypeptide can improve clinical symptoms and laboratory indices, stimulate leukocyte interferon-genesis and normalize humoral and cellular immunity in a juvenile rheumatoid arthritis, rheumatoid arthritis or psoriatic arthritis subject [63].

This invention provides a method for treating or preventing progression of a bronchial asthma in a subject comprising administering an effective amount of the above-described peptide or its functional equivalents or polypeptide. In an embodiment, the said peptide or its functional equivalents or the said polypeptide can reduce the average daily dose of oral steroid required for relief; moderately improve spirometric parameters; and increase sensitivity to dexamethasone in a bronchial asthma subject [11, 36, 38, 64, 65, 66, 67, 68].

This invention provides a method for treating and preventing progression of respiratory infections in a pediatric patient comprising administering an effective amount of the above-described peptide or its functional equivalents or the above-described polypeptide. In an embodiment, the said peptide or its functional equivalents or the said polypeptide can improve immunological indices and decrease the frequency of infections in a pediatric patient with respiratory infection.

This invention provides a method for reducing allergic reactions and drug toxicity in an epileptic subject who uses anticonvulsants comprising administering an effective amount of the above-described peptide or its functional equivalent [6].

This invention provides a method for treating or preventing progression of breast cancer in a subject comprising administering an effective amount of the said peptide or its functional equivalents or the said polypeptide. In an embodiment, the above-described peptide or its functional equivalents or the above-described polypeptide provides immunomodulation by normalizing the levels of the tumor serum marker, CA15.3, and by increasing tumor-infiltrating CD5′ T-cells and CD11 macrophages in a breast cancer subject [60].

This invention provides a method for improving the recovery of a subject after colorectal cancer treatment or surgery comprising administering an effective amount of the above-described peptide or its functional equivalent to the subject [98].

This invention provides a method for inducing the remission of Hodgkin's disease in a subject comprising administering an effective amount of the above-described peptide or its functional equivalent [100].

This invention provides a method for treating or preventing progression of psoriasis in a subject comprising administering an effective amount of the above-described peptide or its functional equivalent to the subject. In an embodiment, the said peptide or its functional equivalents or the said polypeptide can improve clinical symptoms, eradicate rash, relieve pain, and increase activity of immunoregulatory lymphocytes and percentages of CD3+ and CD8 in a psoriasis subject. In another embodiment, the isolated peptide or polypeptide is administered in combination with other therapeutic compounds effective for treating or preventing psoriasis to enhance the efficacy of the isolated peptide or polypeptide of the present invention. Drugs or preparations which can be effectively or synergistically used in combination with LAP include but are not limited to Anthralin, Coal tar, Corticosteriods, Retinoid (Tazarotene), Vitamin D₃ (Calcipotriene), pimecrolimus and tacrolimus [104].

This invention provides a method for treating atherosclerosis and other forms of vascular obstructions in a human subject by administering an effective amount of the above-described peptide or its functional equivalent to the subject.

This invention provides a method for limiting myocardial cell death in a subject by administering an effective amount of the above-described peptide or its functional equivalent to the subject [7, 37].

This invention provides a method for improving the cardiac muscle contractile force reduced by various cardiomyopathy, including hypertension, viral and idiopathic.

This invention provides a method for limiting the rejection reaction that follows organ transplantation in a subject by administering an effective amount of the above-described peptide or its functional equivalent to the subject.

This invention provides a method for treating or preventing progression of HIV or SARS (severe acute respiratory syndrome) infection in a subject by administering an effective amount of the above-described peptide or its functional equivalent to the subject.

An outbreak of atypical pneumonia, referred to as severe acute respiratory syndrome (SARS) and first identified in Guangdong Province, China, has spread to several countries. Similar cases were detected in patients in Hong Kong, Vietnam, and Canada during February and March 2003. The World Health Organization (WHO) issued a global alert for the illness. In mid-March 2003, SARS was recognized in health care workers and household members who had cared for patients with severe respiratory illness in the Fareast. Many of these cases could be traced through multiple chains of transmission to a health care worker from Guangdong Province who visited Hong Kong, where he was hospitalized with pneumonia and died. By late April 2003, over thousands of SARS cases and hundreds of SARS-related deaths were reported to WHO from over 25 countries around the world. Most of these cases occurred after exposure to SARS patients in household or health care settings. This disclosure provides a method to prevent and/or treat SARS.

This invention provides a method for treating or preventing progression of brain cell malfunction due to ischemia and trauma in a subject comprising administering an effective amount of the above-described peptide or its functional equivalent to the subject [17, 24].

This invention provides a method for treating the pathologic consequences of ischemia-reperfusion in a subject by administering an effective amount of the above-described peptide or its functional equivalent to the subject [8].

This invention provides a method for treating any chemical or anesthetic intoxication including but not limited to alcohol and morphine intoxication by administering an effective amount of the above-described peptide or its functional equivalent to the subject.

This invention provides a method for aiding or hastening wound healing in a subject by administering an effective amount of the above-described peptide or its functional equivalent to the subject.

This invention provides a method for treating viral diseases in a subject by administering an effective amount of the above-described peptide or its functional equivalent to the subject.

This invention provides a method for protecting cardiomyocytes from injury by contacting said cardiomyocytes with an effective amount of the above-described peptide or its functional equivalent.

This invention provides a method for protecting cardiomyocytes in a subject by administering to the subject an effective amount of the above-described peptide or its functional equivalent.

In an embodiment the cardiomyocyte is injured. This invention provides a method for protecting cardiomyocytes from further injury by contacting said cardiomyocytes with an effective amount of the above-described peptide or its functional equivalent. Wherein the cardiomyocyte is injured, this invention provides a method for protecting cardiomyocytes in a subject by administering to the subject an effective amount of the above-described peptide or its functional equivalent.

In an embodiment this invention mitigates injuries to cardiomyocytes. This invention provides a method for protecting cardiomyocytes from further injury by contacting said cardiomyocytes with an effective amount of the above-described peptide or its functional equivalent. This invention provides a method for protecting cardiomyocytes from further injury by chemicals or by lack of blood or oxygen in a subject by administering to the subject an effective amount of the above-described peptide or its functional equivalent.

This invention provides a method for the treatment of conditions, injuries and diseases in which apoptosis occurs by administering an effective amount of the above-described peptide or its functional equivalent to the subject [3].

This invention provides a composition capable of inhibiting or killing cancer cells, said composition comprises an effective amount of the above-described peptide or its functional equivalent and a suitable carrier.

This invention provides a method for inhibiting or killing cancer cells by contacting said cancer cells with an effective amount of the above-described peptide or its functional equivalent.

As used herein, cancer cells include but are not limited to breast cancer, bowel cancer, brain cancer, Jurkat cells (the acute T-cell leukemia cell line) [3, 15, 52, 60].

This invention provides a method for inhibiting or killing cancer cells by administering to the subject an effective amount of the above-described peptide or its functional equivalent.

This invention provides a composition containing an amount of the above-described peptide or its functional equivalent which is antagonistic to H1-histamine receptor.

This invention provides a method for producing effects in a cell which are antagonistic to H1-histamine receptors in a cell by contacting said cell with an effective amount of the above-described peptide or its functional equivalent.

This invention provides a method for producing effects which are antagonistic to H1-histamine receptors in a subject by administering to the subject an effective amount of the above-described peptide or its functional equivalent.

This invention provides a composition which is inhibitory to A2-phospholipase activity and which contains an effective amount of the above-described peptide or its functional equivalent in a suitable carrier [39].

This invention provides a method for producing inhibitory A2-phospholipase activity in a cell by contacting said cells with an effective amount of a composition which contains an effective amount of the above-described peptide or its functional equivalent coupled with a suitable carrier (39).

This invention provides a composition for protecting against the effects of Tumor Necrosis Factor (TNF) which contains an effective amount of the above-described peptide or its functional equivalent.

This invention provides a method for protecting against the effects of Tumor Necrosis Factor (TNF) in a cell by contacting said cell with an effective amount of a composition which contains an effective amount of the above-described peptide or its functional equivalent.

This invention provides a method for protecting against the effects of Tumor Necrosis Factor (TNF) in a subject by administering to the subject an effective amount of a composition which contains an effective amount of the above-described peptide or its functional equivalent.

This invention provides a method for treating or preventing the progression of inflammatory bowel disease in a subject comprising administering an effective amount of the above-described peptide or its functional equivalent to the subject.

This invention provides a method for treating or preventing the progression of type 1 diabetes mellitus in a subject comprising administering an effective amount of the above-described peptide or its functional equivalent to the subject.

This invention provides a method for treating or preventing the progression of multiple sclerosis in a subject comprising administering an effective amount of the above-described peptide or its functional equivalent to the subject.

This invention provides a method for treating or preventing the progression of septic shock (Gram negative sepsis) in a subject comprising administering an effective amount of the above-described peptide or its functional equivalent to the subject [62].

This invention provides a method for treating or preventing the progression of Parkinson's Disease in a subject comprising administering an effective amount of the above-described peptide or its functional equivalent to the subject.

This invention provides a method of using the above-described peptide or its functional equivalent to modify the function of sigma 1 and sigma 2 receptors. In one embodiment, the modification can be used to prevent progression of myocardial infarction in a subject. Accordingly, there is provided a method of preventing progression of myocardial infarction in a subject comprising administering to the subject an effective amount of a composition which contains an effective amount of the above-described peptide or its functional equivalent coupled with a suitable carrier.

In another embodiment, the above described modification of sigma 1 and sigma 2 receptors can be used to prevent progression of brain stroke in a subject. Accordingly, there is provided a method for modifying sigma 1 and sigma 2 receptors to prevent progression of brain stroke in a subject comprising administering to the subject an effective amount of a composition which contains an effective amount of the above-described peptide or its functional equivalent.

This invention provides a method for treating or preventing the progression of type 2 diabetes mellitus in a subject comprising administering an effective amount of the above-described peptide or its functional equivalent to the subject.

This invention provides a method for treating or preventing the progression of Alzheimer's in a subject comprising administering an effective amount of the above-described peptide or its functional equivalent to the subject.

This invention provides a method for treating or preventing the progression of amyotrophic lateral sclerosis in a subject comprising administering an effective amount of the above-described peptide or its functional equivalent to the subject.

This invention provides a method for treating or preventing the progression of endo- and exo-toxema and related conditions in a subject comprising administering an effective amount of the above-described peptide or its functional equivalent to the subject.

This invention provides a method for treating or preventing the progression of Crohn's disease (i.e. chronic enteritis) in a subject comprising administering an effective amount of the above-described peptide or its functional equivalent to the subject.

This invention provides a method for treating or preventing the progression of ulcerative colitis in a subject comprising administering an effective amount of the above-described peptide or its functional equivalent to the subject.

This invention provides a method for treating or preventing the progression of hyperthyroidism in a subject comprising administering an effective amount of the above-described peptide or its functional equivalent to the subject [49, 54, 55, 56, 57, 58, 59].

This invention provides a method for treating or preventing the progression of Guillain Barre syndrome in a subject comprising administering an effective amount of the above-described peptide or its functional equivalent to the subject.

This invention provides a method for treating or preventing the progression Systematic lupus erythematosus and other collagen diseases including but not limited to scleroderma in a subject comprising administering an effective amount of the above-described peptide or its functional equivalent to the subject.

This invention provides a method for treating or preventing the activation of Caspases 3, 4, and 8 in a subject comprising administering an effective amount of the above-described peptide or its functional equivalent to the subject.

This invention provides a method for modulating nitric oxide synthase (NOS) in a subject comprising administering an effective amount of the above-described peptide or its functional equivalent to the subject.

This invention provides a method for treating leishmaniasis in a subject by administering to the subject an effective amount of the above-described peptide or its functional equivalent.

This invention provides a method for treating leishmaniasis in a subject by administering to the subject an effective amount of the above-described peptide or its functional equivalent. The above-described peptide or polypeptide may be administered to the subject intramuscularly or subcutaneously. Alternatively, other route of administration may be used.

This invention provides a method of regulating T cell function in a subject comprising administering to the subject an effective amount of a composition comprising the above-described peptide or its functional equivalent.

This invention provides a composition containing an effective amount of the above-described peptide or its functional equivalent in a pharmaceutically acceptable suitable carrier for treatment of leishmaniasis.

This invention provides a composition containing an effective amount of Plaferon-LB in a pharmaceutically acceptable suitable carrier for treatment of leishmaniasis.

This invention also provides a method of treating leishmaniasisin a subject comprising administering to the said subject an effective amount of Plaferon-LB.

The above subject includes but is not limited to mammals. In an embodiment the mammals are dogs or cats.

The invention further provides a process for preparing a pharmaceutical composition which comprises bringing a peptide of the invention into association with a pharmaceutically acceptable excipient or carrier.

This invention provides a substance containing the isolated peptide(s) or polypeptide(s) as described above. In an embodiment, the peptide is conjugated directly or indirectly to another compound. In a further embodiment, the peptide is a protein.

Method for Isolating and Synthesizing the Biologically Active Compounds

The biologically active peptide or polypeptide of the present invention can be synthesized by the process as described below:
(1) obtaining amniotic tissues and incubating the amniotic tissue at 37° C. with 0.01 mg of protein content per 1.0 ml of media;
(2) Inducing the production of the biologically active peptide by amniotic tissues by means of Newcastle Disease Virus (NDV) for 1 hour at 37° C.;
(3) Cultivating the amniotic tissues for 10-12 hours at 37° C.;
(4) Separating the amniotic tissues from the solution containing the biologically active peptide by centrifugation;
(5) Inactivating the NDV by adjusting the pH of the solution to 2.0 and incubating the solution at +4° C. for not less than 3 days;
(6) Purifying the biologically active peptide;

Steps (1) to (6) have been discussed in detail in U.S. patent application Ser. No. 09/928,178 and International PCT Application No. PCT/US01/41666, the contents of which are hereby incorporated in their entireties by reference into this application.
(7) Separating the peptide into high molecular weight (>5000 Da) and low molecular weight (<5000 Da) fractions (See Example);
(8) Testing the fractions for bioactivity;
(9) Decoding the biologically active peptide to determine the amino acid sequence;
(10) Synthesizing the peptide or polypeptide using the decoded amino acid sequence; and
(11) Testing the synthesized peptide or polypeptide for bioactivity.

This invention provides a compound or peptide produced by the process as described above.

This invention will be better understood from Examples which follow. However, one skilled in the art will readily appreciate that the specific methods and results discussed are merely illustrative of the invention as described more fully in the claims which follow thereafter.

### EXPERIMENTAL DETAILS

### Example 1

Biologically Active Peptides in the Low Molecular Weight Fractions of Plaferon-LB.

Low molecular weight components of Plaferon-LB (PM<5000 Da) were separated from high molecular weight components of Plaferon-LB (PM>5000 Da) using size exclusion chromatography (Sephadex G25) (FIG. 1).

The fractions containing the high molecular weight (>5000 Da) compounds and the fractions containing the low molecular weight (<5000 Da) compounds were pooled and freeze dried.

The two freeze dried pools were analyzed by SE and RP-HPLC (FIG. 2a & 2b). RP-HPLC analysis confirmed the results obtained and several peptides were detected in the low molecular weight fraction (FIG. 3).

The low molecular weight components of Plaferon-LB were further fractionated into 9 fractions, referred to herein as Fractions 0-8, using reverse phase chromatography (RP-Chromatography) (FIG. 4) and these 9 fractions were separately tested on the mouse lipopolysaccharide (LPS) sepsis model for bioactivity. Biological activity was found in Fractions 2, 3 and 4, the Fraction 4 being the most active

Materials and Methods

Five to six weeks old female CD1 mice (Charles River, Calco, Italy) were allowed to adapt one week to their environment before commencing the study. They were kept under standard laboratory conditions with ad libitum food and water. The mice were injected i.p. with 1 mg LPS (Sigma Chimica, Milan, Italy). Mortality was recorded every 24 hours up to 72 hours after challenge with LPS.

Results and Conclusions

As expected, 100% lethality was observed within 72 hours from LPS injection in control mice treated with PBS (Table 1). In contrast, the prophylactic treatment of the mice with 0.5 ml/mouse of Fraction 2, 3, or 4 given at −24 and −1 hour prior to LPS significantly reduced the cumulative rate of lethality (Table 1). Fraction 4 afforded the best protective effect. The data from this experiment indicated that the Fractions 2 and 3 of the low molecular weight fractions of PLB are also capable of exerting protective action in LPS-induced lethality. The optimal effect is seen when the fraction is administered at 0.5 ml/mouse at −24 and −1 prior to LPS.

TABLE 1
Efficacy of Plaferon-LB low molecular weight subtractions
to counteract LPS-induced lethality in mice

| Treatment | Time of | Lethality | Lethality | Lethality |
|---|---|---|---|---|
| (0.5 ml) | administration* | 24 h | 48 h | 72 h |
| PBS (control) | −24, −1 | 25% | 75% | 100% |
| | | (5/20) | (15/20) | (20/20) |
| PLB fraction 0 | −24, −1 | 25% | 75% | 75% |
| | | (5/20) | (15/20) | (15/20) |
| PLB Fraction 1 | −24, −1 | 15% | 75% | 100% |
| | | (3/20) | (15/20) | (20/20) |
| PLB Fraction 2 | −24, −1 | 15% | 35% | 35% |
| | | (3/20) | (7/20) | (7/20) |
| PLB Fraction 3 | −24, −1 | 15% | 35% | 35% |
| | | (3/20) | (7/20) | (7/20) |
| PLB Fraction 4 | −24, −1 | 25% | 25% | 25% |
| | | (5/20) | (5/20) | (5/20) |
| PLB Fraction 5 | −24, −1 | 15% | 35% | 50% |
| | | (3/20) | (7/20) | (10/20) |
| PLB Fraction 6 | −24, −1 | 0% | 65% | 65% |
| | | (0/20) | (13/20) | (13/20) |
| PLB Fraction 7 | −24, −1 | 50% | 90% | 100% |
| | | (10/20) | (18/20) | (20/20) |
| PLB fraction 8 | −24, −1 | 0% | 65% | 75% |
| | | (0/20) | (13/20) | (15/20) |

*Hours relative to LPS administration

There appears to be some bio-activity in Fractions 0, 5, 6 and 8.

Although, this experiment showed that Fractions 2, 3 and 4 of the low molecular weight fractions of Plaferon-LB are capable of exerting the best protective action in LPS-induced lethality, other fractions of the low molecular weight fraction of Plaferon-LB (i.e. Fractions 0, 5 and 6) also exhibited some bio-activity. It is believed that Fractions 0, 1, 5, 6, 7 and 8 of the low molecular weight fractions of Plaferon-LB and the untested high molecular weight fractions of Plaferon-LB contain biologically active peptides which have identical/similar therapeutic and pharmacological properties as the biologically active peptides found in Fractions 2, 3 and 4 of the low molecular weight fractions of Plaferon-LB.

### Example 2

Bio-active Peptide in Fraction 2, 3 and 4 of the Low Molecular Weight Fraction of the Plaferon-LB

Fraction 2, 3 and 4 of the low molecular weight components of Plaferon-LB were characterized by mass spectrometry analysis (MALDI-TOF).

Materials and Methods

Mass Spectrometry (MALDI TOF)

Fraction 2, 3 and 4 after preparative reverse phase chromatography were analyzed by MALDI TOF Mass spectrometry using a Voyager System 1178 (Applied Biosystem):

Matrix: 3-hydroxypicolinic acid

Mode of operation: linear

Polarity: positive

Acquisition control: manual

Accelerating voltage: 23000V

Grid voltage: 95%

Extraction delay time: 400 nsec

Acquisition mass range: 500-20000 Da

Number of laser shots: 25/spectrum

Laser intensity: 1820

Results

Mass Spectrometry (MALDI-TOF)

Table 2 below summarizes the results obtained.

TABLE 2
Summary of the mass of the peptides
detected in Fraction 2, 3 and 4

| Fraction 2 | Fraction 3 | Fraction 4 |
|---|---|---|
| 664.9 | 664.7 | 673 |
| 825.6 | 826.5 | |
| 986.6 | 986 | |
| 1148 | 1147 | |
| 1307.9 | | |
| 1468.3 | | |

Fraction 2 contained one main peptide of 664.9 Da and multiple (5) additions of approximately 160 Da. Fraction 3 contained the same peptide with three additions of 160 Da and Fraction 4 contained the same peptide without any additions. The addition of 160 Da is consistent with phosphorylation (2×80 Da).

The sequence of the biologically active peptide in Fraction 4 was determined using mass spectrometry (FIG. 5). Table 3 below summarizes the mass spectrometry results obtained from Fraction 4.

TABLE 3

Summary of the mass of the peptides detected in
Fraction 4

| | |
|---|---|
| Mass (Da) | Amino Acid Sequence |

| | |
|---|---|
| 785.5 | NVAAVEIA |
| | (SEQ ID NO. 3) |
| 801.5 | NVSAVEIA |
| | (SEQ ID NO. 1) |
| 837.4 | NVS(+phosphate)AVEIA(−CO2) |
| | (SEQ ID NO. 2) |
| 881.4 | NVS(+phosphate)AVEIA |
| | (SEQ ID NO. 2) |
| 917.5 | NVCKVEIA (+2Na) |
| | (SEQ ID NO. 4) |
| | NVFKVEIA |
| | (SEQ ID NO. 5) |
| 1243.8 | NVS(+2xGalNAc or GlcNAc, +phosphate)AVEIA |
| | (−CO2) |
| 1287.8 | NVS(+2xGalNAc or GlcNAc, +phosphate)AVEIA |
| 1331.9 | NVS(+2xGalNAc or GlcNAc, +phosphate)AVEIA |
| | (+2Na) |
| 1375.9 | |
| 1419.9 | |
| 1463.9 | |
| 1508.0 | |
| 1552.0 | |
| 1596.0 | |
| 1640.0 | |
| 1127.7 | |
| 1171.7 | |
| 1215.7 | |
| 1259.8 | |
| 1303.8 | |
| 1347.8 | |
| 1391.9 | |
| 1435.9 | |
| 1480.0 | |
| 1523.9 | |
| 1568.0 | |

### Example 3

Bioactivity of Chemically Synthesized Lajor Active Peptide (LAP)

Synthetic peptide or Lajor Active Peptide (LAP) was synthesized chemically to produce the amino acid sequence of the previously-identified bioactive peptide contained in Fractions 2, 3 and 4 of the low molecular weight components of PLB. The efficacy of the LAP was evaluated using the same experimental conditions under which the Fractions 2, 3 and 4 of the low molecular weight components of Plaferon-LB were found to be effective (See Example 1). Mice treated with Fraction 4 prepared from PLB were used as positive controls.

Materials and Methods

Six weeks old female CD1 mice (Charles River, Calco, Italy) were used. The mice were allowed to adapt one week to their environment before commencing the study. They were kept under standard laboratory condition with ad libitum food and water.

The mice were injected i.p. with 1 mg of lipopolysaccharide (LPS) (Sigma Chimica, Milan, Italy). Mortality was recorded every 24 hours up to 72 hours after challenge with LPS.

Results

As expected, 100% lethality was observed within 72 hours from LPS injection in control mice treated with PBS (Table 4). In contrast, the prophylactic treatment of the mice with 0.5 ml/mouse of Fraction 4 given at −24 and −1 hour prior to LPS significantly reduced the cumulative rate of lethality (Table 4). Moreover, both the kinetic and cumulative rate of lethality were unaffected by the different doses of LAP tested, the so-treated mice exhibited a kinetic and cumulative incidence of LPS-induced lethality very similar to that of controls.

The cumulative rate of lethality was also markedly reduced by the prophylactic treatment with 10 ucg of the LAP.

The data from this experiment indicate that LAP possesses powerful immunomodulatory and protective action in a murine model of LPS-induced lethality that is comparable to that obtained with Fraction 4.

This study confirms the bioactivity of the peptide and its potential use in several immuno-inflammatory or auto-immune diseases such as type 1 diabetes, multiple sclerosis, Guillain Barrè syndrome, chronic hepatitis etc. Studies in preclinical models of immuno-inflammatory or auto-immune diseases are warranted to provide “in vivo” proof of concept. The beneficial effects observed with natural (unfractionated) PLB in preclinical models of multiple sclerosis, type 1 diabetes, rheumatoid arthritis and inflammatory hepatitis seem to anticipate a similar beneficial role for the LAP in these conditions.

TABLE 4
Test of potency of the efficacy of LAP in counteracting
lethality induced by a DL100 dose (1 mg/mouse) of LPS

| Treatment | Time of | Lethality | Lethality | Lethality |
|---|---|---|---|---|
| (0.5 ml) | Administration* | 24 h | 48 h | 72 h |
| Vehicle | −24, −1 | 15% | 80% | 100% |
| | | (3/20) | (16/20) | (20/20) a |
| PLB | −24, −1 | 5% | 20% | 40% |
| Fraction 4 | | (1/20) | (4/20) | (8/20) b |
| Peptide | −24, −1 | 10% | 35% | 80% |
| 2.5 mcg | | (2/20) | (7/20) | (16/20) |
| Peptide | −24, −1 | 10% | 35% | 50% |
| 10 mcg | | (2/20) | (7/20) | (10/20) c |

*Hours relative to LPS administration; b vs. a, p > 0.0001; d vs. a, p = 0.001; c vs. b = not significant by chi-square

The data presented herein (or below) demonstrated that Plaferon-LB (PLB) exhibited clear-cut beneficial effects in 5 different rodent models of human immunoinflammatory/auto-immune diseases such as MS (PR-EAE in DA rats), gram-negative sepsis (LPS-induced lethality), chronic active hepatitis (Concanavalin A-induced hepatitis), rheumatoid arthritis (oil-induced arthritis) and type 1 diabetes mellitus (NOD mouse model).

This synthetically active peptide or Lajor Active Peptide (LAP), comprising amino acid sequence NH2-NVSAVEIA-COOH (SEQ ID NO.1), manufactured chemically or using recombinant DNA technology is believed to possess similar/identical therapeutic and pharmacological properties as the biologically active peptide Fraction 4(See Example 5) isolated from Plaferon-LB. Therefore, studies that will be undertaken to investigate the effects of LAP in rodent models of various human diseases and injuries will exhibit similar beneficial effects as found using Plaferon-LB.

Studies have proven the effects of PLB in preclinical models of type 1 diabetes mellitus (NOD mouse), multiple sclerosis (DA rat EAE) immunoinflammatory hepatitis (Con A-induced hepatitis in mice), rheumatoid arthritis (oil-induced arthritis in DA rats) and sepsis (murine endotoxemia). And that these data provide valuable proof of concepts for the efficacy of PLB in these auto-immune diseases. Because these diseases are all characterized by up-regulated synthesis/function of type 1 pro-inflammatory cytokines (TNF-α, IL-2, IFN-gamma), one possible mode of action of PLB may rely on specific antagonism of these cytokines. In addition, because these cytokines are also pathogenetically involved in other human immunoinflammatory or auto-immune diseases such as Hashimoto's thryoiditis, Crohn's disease, psoriasis and Guillain Barre syndrome it is believed that PLB may also be considered for the treatment of these disorders. (Also See National Institutes of Health Autoimmune Diseases Coordinating Committee Autoimmune Diseases Research Plan, <http://www.niaid.nih.gov/dait/pdf/ADCC_Report.pdf>, the contents of which are incorporated in its entirety by reference into this application)

### Example 4

Effects of PLB Prophylaxis on the Course of PR-EAE in DA Rats

A condition resembling MS, experimental allergic encephalomyelitis (EAE), can be induced in susceptible strains of mammalian species, by immunization with CNS antigens in appropriate adjuvant (69). A major drawback of most EAE models, such as EAE in Lewis rat, and which make important clinical and histological differences with the human disease counterpart, is the occurrence in these rats of a monophasic disease with rare or absent demyelination. However, a severe, protracted relapsing and demyelinating form of EAE (PR-EAE) has recently been reported to be inducible in DA rats by immunization with either syngeneic or guinea pig spinal cord emulsified in incomplete (FIA) or complete Freund's adjuvant (FCA) (70). Therefore, this model offers a unique in vivo tool for studying immune-mediated mechanisms involved in the generation of chronicity and demyelination and to study novel immunotherapeutical approaches to be considered for the treatment of human MS.

The effects of PLB prophylaxis on the course of PR-EAE in DA rats was evaluated.

Materials and Methods

Animals

Males DA rats, (Harlan Nossan, Italy), weighing 230-270 g. were used for the study.

Immunization

Fifty μg guinea pig spinal cord (Sigma St. Louis Mo.), minced thoroughly were emulsified with 100 μl of FIA, (Sigma.) and 2 μgs Mycobacterium tuberculosis, strain H 37 RA (Difco, Detroit, Mich.) and were injected subcutaneously (s.c.) at the base of the tail.

Treatment

Plaferon-LB was dissolved in 10 ml of sterile saline and then injected i.p. at the dose of 0.5 ml/rats five consecutive days a week. Treatment was started one day prior to immunization and it was continued until day 40 post immunization. One ampoule of Plaferon-LB was dissolved in 10 ML of PBS and each rat received daily 0.5 ml of the drug (i.p.) six times a week.

Clinical Scoring

The rats were weighed every day and clinical signs scored by an observer unaware of treatment regimen as described elsewhere (71). The clinical score was as follows: 0=no illness; 1=flaccid tail; 2=moderate paraparesis, 3=severe paraparesis, 4=tetraparesis, 5=death.

Results

Lack of Toxicity of Prolonged PLB-Treatment

Treatment with PLB was well tolerated as judged from the behavior and clinical appearance of the rats, and no clinical signs of toxicity could be observed.

Prophylactic Treatment with PLB Ameliorates the Clinical Course of PR-EAE in DA Rats

As expected, throughout the 50 days observation period, classical signs of PR-EAE were observed in the group of rats (15/15) treated with PBS (FIG. 6); As previously reported (71), variable protracted disease followed after the first attack, with some rats showing remission of clinical signs and up to two or more relapses (FIG. 6); To evaluate whether PLB influenced the course of PR-EAE, DA rats were treated with this drug under an early prophylactic regimen one day prior to immunization until day +40 post immunization. Although the cumulative incidence of EAE among PLB-treated rats (16/18, 88.9%) was not significantly different from that of control rats, relative to these latter animals, those treated with Plaferon-LB exhibited a milder course of the disease entailing lower EAE cumulative score and subsequent relapses of shorter duration and reduced severity. Those data was confirmed by two independent experiments. Because the data were highly reproducible in the two studies, they were merged and shown here as a single study (FIG. 6).

### Example 5 (Also See Example 3)

Effects of Plaferon-LB in Murine Lipopolysaccharide (LPS)-Induced Lethality, a Model of Human Endotoxemia

This study uses LPS as a model for human sepsis to show the efficacy of Plaferon-LB in endotoxaemia.

Type 1 cytokines, such as interleukin (IL)-1, IL-12, tumor necrosis factor (TNF)-α and interferon (IFN)-γ, and type 2 cytokines, such as IL-6 and IL-10 (72) play a pivotal role in the pathogenesis of endotoxic shock conditions through their proinflammatory and vasoactive properties (71). However, the production and the action of type 1 cytokines may be antagonized by type 2 anti-inflammatory cytokines and the balance between these two cytokine subsets may therefore influence the host response to endotoxaemia (73). Thus, lipopolysaccharide (LPS)-induced lethality in mice is prevented by blockade of endogenous IL-1, IL-12, TNFα or IFNγ with specific antagonists or by administration of type 2 cytokines, such as IL-4, IL-10 or IL-13 (74-78). Pharmacological compounds capable of inhibiting the production/action of type 1 cytokines while at the same time up-regulating the production of type 2 cytokines may therefore be suitable candidates for the prevention/treatment of endotoxaemia.

These observations prompted us to evaluate here the effects of Plaferon-LB on the course of experimental lethal endotoxaemia in mice. This condition, which can be induced by the injection with a single high-dose of LPS shares some immunological and pathogenic pathways similar to human endotoxemia and is and has been extensively used as an in vivo model to understand the pathogenic mechanisms and evaluate novel immuno-therapeutical approaches for the treatment of the syndrome.

The data showed that PLB successfully counteracted LPS-induced lethality in mice regardless of whether it was given prior to or 1 hour after endotoxin challenge.

Materials and Methods

Reagents

PLB was produced as described elsewhere (see U.S. patent application Ser. No. 09/928,178, filed Aug. 9, 2001, and Patent Cooperation Treaty (PCT) Application Number, PCT/US01/41666, filed Aug. 9, 2001 with International Publication Number WO 02/12444). It was dissolved in 10 ml PBS and administered to the mice at either 0.5 or 1 ml i.p. LPS (serotype 0127:B8) was purchased from Sigma Chemicals (St. Louis, Mo., USA) and sterile water for injection from a local pharmacy.

Mice

Four to 6 weeks old female CD1 mice were purchased from Charles River (Calco, Italy)

Experimental Design

All animal procedures were in accordance with the institutional guidelines of the University of Catania, which are in compliance with national laws for the Care and Use of laboratory animals. To induce lethal endotoxaemia, the mice were injected i.p. with 3 mg LPS diluted in 0.3 ml water for injection. This dose of LPS was selected on the basis of previous experiments showing its capacity to induce lethality within 3 days in 75 to 100% of the mice.

The effects of PLB on the development of LPS-induced lethality were evaluated both under a prophylactic and “early therapeutic” regime. For prophylaxis, the mice received i.p. injections with either 0.5 or 1 ml PLB, 24 hours and 1 hour prior to LPS-challenge (Table 5). Control mice were treated under similar conditions with PBS alone. The “therapeutic” capacity was tested by treating the mice with a single i.p. injection of 1 ml PLB given 30 minutes after LPS (Table 5).

In addition, a positive control group of mice consisted of animals given a polyclonal anti-murine TNF-α (Peprotech, UK) antibody (Ab) that is known from our work and literature data to counteract the lethal action of LPS when given under prophylactic but not therapeutic conditions. Lethality was assessed at 1 day intervals for 3 consecutive days.

Statistics

Cumulative lethalities at 72 hours after LPS injection were compared using chi-square P values equal or lower than 0.05 were considered significant.

Results

Effect of Prophylactic Treatment with PLB on LPS-Induced Lethality

As expected, all the control mice (15/15) died within 3 days of LPS-injection (Table 5). In contrast, prophylactic treatment with 0.5 ml PLB given at −24 and −1 hour prior to LPS significantly improved the survival of the mice, with only 10/15 of the mice, 66.7%) dying during the observation period (Table 5). PLB did not merely delay the lethal action of LPS, as none of the remaining mice from the controls or from the PLB-treated group died during a follow-up period of one week. Prophylacitc treatment of the mice with anti-TNF-α polyclonal antibody yielded afforded a protective effect similar to that observed with PLB, 9/15 (60%) of the so-treated group being dead by 72 hours after LPS injection with a kinetic of mortality very similar to that observed with PLB-treatment (Table 5).

Effect of “Early Therapeutic” Treatment with PLB on LPS-Induced Lethality

To evaluate whether PLB also had a therapeutic capacity, experiments were carried out where the drug was first administered to the mice 30 minutes after they had been injected with LPS. As shown in Table 5, “therapeutically-administered” PLB also diminished LPS-induced lethality. The cumulative incidence of mortality was 100% in PBS-treated controls (15/15) and 62.5% (10/15) in the PLB-treated mice. Again, none of the mice died during the one week follow-up period. In contrast, administering anti-TNF-α Ab 30 minutes after LPS failed to counteract the lethal effects of the endotoxin, the cumulative incidence of mortality observed in this group (15/15, 100%) being identical to that of control mice challenged with LPS and treated with PBS.

TABLE 5
Effects of time of administration of
PLB on LPS-induced lethality in mice

| | Dose/mouse | | | | | | | |
|---|---|---|---|---|---|---|---|---|
| | Time of adm. | | | | Number of dead | | | |
| Treat (n) | relative to LPS | | | | 24 h | 48 h | 72 h | Total |
| PBS (15) | 0.5 | ml | −24, | −1 h | 6 | 9 | 0 | 15 |
| PLB (15) | 0.5 | ml | −24, | −1 h | 4 | 5 | 1 | 10* |
| Anti-TNF-α | 0.5 | mg | −24, | −1 | 4 | 3 | 2 | 9** |
| Ab (15) | | | | | | | | |
| PBS (15) | 1 | ml | +30 | min | 5 | 9 | 1 | 15 |
| PLB (15) | 1 | ml | +30 | min | 4 | 4 | 2 | 10* |
| Anti-TNF | 0.5 | mg | +30 | min | 5 | 7 | 3 | 15 |
| αAb (15) | | | | | | | | |

**p < 0.05 by chi-square
*p = 0.05 vs. PBS-treated controls by chi-square

### Example 6

Protection from Concanavalin A-Induced T-Cell Dependent Hepatic Lesions and Modulation by Plaferon-LB

Recently, a new model of hepatitis has been described which can be induced in mice by a single i.v. injection of Concanavalin (Con) A (79-81). Within 8-24 hours (h), clinical and histological evidence of hepatitis occur with elevation of transaminase activities in the plasma and hepatic lesions characterized by massive granulocyte accumulation and hepatic necrosis (79-81). Con A-induced hepatitis is both T-cell and macrophage dependent; it can not be induced in nude athymic mice lacking immunocompetent T cells, and it is prevented by anti-T cell immunosuppressants such as cyclosporin A (CSA) and FK506, or by blockade of macrophage functions with silica particles (79-81).

The precise mechanism(s) by which T cells and macrophages exert their hepatogenic potential is not known. Because a massive release of macrophage and T-cell derived cytokines (IL-1, IL-2, IL-6, IL-10, TNF-α, IFN-γ gamma and GM-CSF) occurs with different kinetics in response to ConA, a role has been envisaged for these cytokines in the development of the hepatic lesions. Nonetheless, the role of cytokines in the pathogenesis of this immunoinflammatory condition remains to be defined. For example, the disease is equally prevented by specific inhibitors (monoclonal antibody, soluble receptors) of-TNF-α, IL-4, IFN-gamma IL-12 antibody (Ab) as well as by exogenously-administered IL-6 and IL-10 and the outcome of the disease may therefore depend on a fine balance between pro- and antiinflammatory cytokines released by ConA-activated cells (79-81).

The effects of PLB on the Con-A induced hepatic lesions has been tested. The data clearly show that the drug is effective in preventing histological and serological signs of hepatitis regardless of whether it is given prophylactically (prior to ConA) or therapeutically (after ConA).

Material and Methods

Reagents

PLB was produced as described elsewhere (see U.S. patent application Ser. No. 09/928,178, filed Aug. 9, 2001, and Patent Cooperation Treaty (PCT) Application Number, PCT/US01/41666, filed Aug. 9, 2001 with International Publication Number WO 02/12444). It was dissolved in 10 ml PBS and administered to the mice at 0.5 ml i.p. CSA (Novartis, Basle, Switzerland) was bought from a local pharmacy, diluted at the desired concentration in sterile olive oil and injected i.p. at the dose of 100 mg kg. bd wt. Con A was purchased from Sigma Chemicals (St. Louis, Mo., USA) and sterile water for injection from a local pharmacy.

Mice and Hepatitis Induction

Six to eight weeks old male Naval Medical Research Institute (NMRI) male mice were purchased from Charles River, Calco, Italy

The food was withdrawn 16 h prior to the experiments. The mice were divided into 3 experimental groups and challenged each with 20 mg/Kg. Con A. Con A was dissolved in sterile phosphate buffered saline (PBS) and injected to mice via the tail vein. Three groups were treated i.p. with PBS (Sigma Chemical), PLB or, as positive control, with CSA according to the experimental design shown in the Table. The latter group was used as a positive control group as previous data have shown its ability to prevent Con A-induced hepatitis (79). An additional control group consisted of mice challenged only with PBS (See Table 6).

Because marked increases of transaminase activities along with severe histological signs of hepatic injuries have been reported to develop 8 h after Con A injection in these mice (79-81), the animals were sacrificed after 8 hour, and blood and livers were collected.

TABLE 6
Experimental design and effects of PLB on ConA-induced hepatitis in mice

| | | Hepatitis | | | | | |
|---|---|---|---|---|---|---|---|
| | | induction | Treatment | | | ALT | |
| Groups | N° | (Con A) | −24 h | +1 h | −1 h | (U/ml) | p |
| A | 15 | − | — | — | — | 40.7 ± 16.7 | <0.0001 |
| B | 12 | + | PBS | PBS | PBS | 3645 ± 2068 | control |
| C | 15 | + | PLB* | PLB* | — | 348 ± 269.9 | <0.0001 |
| D | 15 | + | | | PLB* | 294.6 ± 116.6 | <0.0001 |
| E | 15 | + | — | CSA** | — | 319.3 ± 161 | <0.0001 |
| F | 14 | + | — | — | CSA** | 3795 ± 2497 | n.s. |

*PLB was given at 0.5 ml/mouse and
**CSA at 100 mg/kg/mouse.

Eight hours after Con A-application the mice were sacrificed and blood samples collected from individual mice for ALT measurement.

For statistical analysis each group is compared to group B.

Assay for Plasma Transaminase Activities

Plasma alanine aminotransferase (ALT) activity was determined by a standard photometric assay using a bichromatic analyzer.

Calculation of Data

Results are expressed as mean values ±SD. Statistical analysis was performed by ANOVA.

Results

PLB-Induced Protection against Serological and Histological Signs of ConA-Induced Hepatic Injury

Three out of 15 (20%) of Con A/PBS-treated control mice, and 1 of 15 (6.7%) of those challenged with Con A and treated under a therapeutic treatment with CSA died before sacrifice. These mice were not considered for serological analyses.

As expected, and in agreement with previous studies (79-81, 91-94), acute signs of liver damage mirrored by marked elevations of ALT in the plasma were found in PBS-treated control mice within 8 hours after challenge with Con A. In contrast, both CSA and PLB reduced in a highly significant fashion and at a comparable extent the increase in ALT values induced by Con A when administered upon a “prophylactic” regime prior to Con A-challenge (Table 6). However, only PLB, but not CSA, inhibited development of hepatitis when administered upon a “therapeutic” regime after Con A-application (See Table 6).

Although histological analyses were not performed in this preliminary set of experiments, ALT values are known to correlate in this model to the extent of inflammatory infiltrations of the liver and to the hepatocytic necrosis. (79-81) It seems therefore likely that the diminished blood levels of ALT observed in PLB (and CSA)-treated mice may be associated to reduced inflammatory infiltration of the liver and inhibition of necrotic and apoptotic pathways of hepatocyte damage and death.

### Example 7

Inhibition of Oil-Induced Arthritis in DA Rats by Plaferon-LB Prophylaxis

Oil-induced arthritis (OIA) is an inflammatory and self-limiting polyarthritis that can be induced in DA rats by subcutaneous injection of mineral oil such as incomplete Freund's incomplete adjuvant (82-84). The joints are initially mainly infiltrated by polymorphonuclear cells but monocytic cells are also present. The disease is T-cell dependent as it is prevented and cured by inhibiting T cell function with monoclonal antibodies directed against the T cell receptor (82) and it can be transferred by CD4+ T cells belonging to the Th1 subtype (83). Like in human RA, TNF-α also seems to play a major pathogenetic role in DA rats OIA (84). OIA thus provide a suitable in vivo tool for studying immunopathogenic mechanisms of and new immunopharmacological approaches for the treatment of human RA.

The results of this study provide evidence that Plaferon-LB prophylaxis favorably influences the course of OIA in rats.

Materials and Methods Animals

Ten to 12 week-old female DA rats purchased from Harlan Nossan (Udine, Italy) were used for the study. The rats were kept under standard laboratory conditions (non-specific pathogen free) at the animal house of the Department of Biomedical Sciences of the University of Catania (Italy). They had free access to food and water and were allowed to adapt at least one week to their environment before commencing the study.

Induction of OIA and PLB Prophylaxis FIA (Difco, Detroit, Mich., USA) was emulsified with phosphate buffered saline (PBS) pH 7.4, 1: v/v and 200 ul was injected subcutaneously at the base of the tail under light ether anesthesia.

Plaferon-LB was produced as described elsewhere (see U.S. patent application Ser. No. 09/928,178, filed Aug. 9, 2001, and Patent Cooperation Treaty (PCT) Application Number, PCT/US01/41666, filed Aug. 9, 2001 with International Publication Number WO 02/12444). It was dissolved in 10 ml saline and administered to the rats (n=20) at 0.5 ml i.p. Treatment was started one day prior to FIA-challenge and continued six times weekly until day 30 after FIA injection. After drug withdrawal the rats were evaluated another 10 days to evaluate for eventual flare-up of arthritis. The control group of animals (n=20) was constituted of rats treated under the same experimental conditions with PBS. Each group consisted of 20 rats.

Evaluation of Arthritis

During the study period and to 40th day after FIA-challenge, arthritis was assessed every other day by an observer unaware of the treatment of the rats using a scale from 0 to 16, each of four paws scored from 0-4 where 0=no arthritis, swelling of the ankle 1 point; swelling of one or more intratarsal and/or metatarsal joints, 1 point; and swelling of one or more intraphalangeal joints, 1 point; 4=swelling of all joints, i.e. the entire paw.

Results

Plaferon-LB Prophylaxis Prevents OIA-Induced Arthritis in DA Rats

100% of PBS-treated control rats injected with a single dose of 200 ul FIA in DA rats developed OIA. The initial signs of disease were observed 11-14 days after FIA-injection (FIG. 7), most often appearing as symmetrical swelling of the metatarsophalangeal or ankle joints of the hind paws. The arthritis subsequently involved the entire hind paw; frontal joints also became inflamed late during the course of the disease. It progressively declined up to complete recovery starting from around day 30 after FIA-challenge (FIG. 7).

The course of OIA-arthritis was favorably influenced by PLB-prophylaxis, the treated rats exhibiting a markedly milder course of the disease that was mirrored by a significantly lower (p<0-0001) arthritic score than that recorded in control rats (FIG. 7). PLB was apparently well tolerated by the rats as judged by their behavior and appearance. No differences in body weights could be observed between PLB- and PBS-treated control rats at the end of the study (FIG. 8).

### Example 8

Prevention of Spontaneous Auto-Immune Diabetes in NOD Mouse by Plaferon-LB Prophylaxis

The NOD mouse serves as one of the best characterized and most widely used models of auto-immune diabetes (85-89). Like in the human disease counterpart, the clinical development of hyperglycaemia is temporarily associated with the selective inflammatory infiltration of the pancreatic beta-cells from T cells and macrophages (85-89). The T-cell and macrophage-dependent nature of NOD mouse diabetes is proven by the possibility to fully prevent its development by targeting the function of these cells with monoclonal antibodies, silica particles (that are toxic for macrophages) or anti-T cell drugs such as CSA (85-89). The cumulative incidence of disease is reached by the age of 7-8 months and it may vary from colony to colony from 60 to 80%, and females have a higher incidence of diabetes than males (85-89). In a similar fashion to human type 1 DM, NOD mice develop insulitis long before the onset of overt diabetes, often starting in a slowly progressive way from the age of 4-5 weeks (85-89).

In this study we have evaluated the effects of prolonged prophylaxis treatment with PLB on the development of spontaneous insulitis and auto-immune diabetes in female NOD mice.

Materials and Methods

Reagents

PLB was produced as described elsewhere (see U.S. patent application Ser. No. 09/928,178, filed Aug. 9, 2001, and Patent Cooperation Treaty (PCT) Application Number, PCT/US01/41666, filed Aug. 9, 2001 with International Publication Number WO 02/12444). It was dissolved in 10 ml PBS and administered to the mice at 0.5 ml i.p. CSA (Novartis, Basle, Switzerland) was bought from a local pharmacy, diluted at the desired concentration in sterile olive oil and given by gavage at the dose of 25 mg kg. bd wt. PBS was purchased from Sigma-Chimica (Milan, Italy).

Animals

Five to 6 weeks-old female NOD mice were purchased from Charles River (Calco, Italy).

Experimental Design

Euglycaemic female NOD mice were randomly allocated into 3 different groups receiving PLB, PBS or CSA according to the experimental design shown in the Table. PBS-treated mice served as controls for PLB-treated mice while CSA-treated mice constituted the “positive” control group as it has been previously demonstrated that when administered upon the treatment regime used in this study (Table 7) CSA successfully prevents development of both insulitis and diabetes in NOD mice (90).

Treatment was started between the 5^{th} and 6th week of age. Because insulitis is virtually absent in NOD mice at this age (85-89), this approach allowed us to investigate the effects of PLB-treatment in the early diabetogenic pathways of NOD mouse diabetes.

Treatments were given until the age of 20 weeks. During the study period the mice were screened for diabetes development twice a week by means of glycosuria followed, when positive, by measurement of glycaemia. Mice were diagnosed as diabetics when fasting glycaemia was above 11.8 mmol/l for 2 consecutive days. At the end of the study period the remaining euglycaemic mice from the different groups were sacrificed and pancreata specimens collected for the severity of insulitis.

Histological Examination of Pancreatic Islets

This was performed in a blind fashion by an observer unaware of the treatment or the status of the mice as described in detail elsewhere. At least 10 islets were counted for each pancreas. The degree of mononuclear cell infiltration was graded as follows: 0, no infiltrate; 1, periductular infiltrate; 2, periislet infilrate; 3, intraislet infiltrate; 4, intraislet infiltrate associated with beta cell desctruction. The mean score for each pancreas was calculated by dividing the total score by the numbers of islets examined.

Results

Early Prophylactic Treatment with PLB Prevents Insulitis Development and Reduces the Cumulative Incidence of Diabetes in NOD Mice

An acute form of diabetes with glycosuria and hyperglycaemia occurred in a large number (9/20, 45%) of PBS-treated control NOD mice by the age of 20 weeks. In contrast, the cumulative incidence of diabetes was significantly reduced both by CSA (2/20, 10%) and, at an even greater extent, by PLB that completely suppressed development of disease (0/20) (See Table 7).

In agreement with these clinical data, histological analysis of pancreatic beta cells from these groups of mice revealed that both CSA and PLB significantly milded the insulitis process as compared to PBS-treated control animals. So, while most of these latter mice showed actively ongoing insulitis varying from periislet infiltrate to intraislet infiltrate associated with beta cell destruction, both CSA- and PLB treated mice mostly exhibited an insulitis process characterized from periductular infiltrate or periislet infiltrate. This resulted in an insulitis score that was significantly lower than that of PBS-treated control mice (Table 7). No significant differences could be noticed in the insulitis score between PLB-treated and CSA-treated NOD mice (Table 7).

TABLE 7
PLB prophylaxis prevents development of insulitis
and auto-immune diabetes in NOD mice

| | | Incidence | Insulitis |
|---|---|---|---|
| Groups (n) | Treatment | of diabetes | score |
| A (20) | PBS | 9/20 (45%) ^{a} | 2.3 ± 1.2^{d} |
| B (20) | CSA | 2/20 (10%) ^{b} | 1.1 ± 0.8^{e} |
| C (20) | PLB | 0/20^{c} | 1 ± 0.9^{f} |

Five to 6 weeks old euglycaemic female NOD mice were treated with PBS (0.5 ml), or PLB (0.5 ml) or CSA (25 mg/kg. bd wt. via gavage) until the age of 20 weeks. PBS and PLB were administered i.p. 6 times a week and CSA was given through gavage on alternate days. Diabetes was diagnosed as described in the M&M section. Diabetic mice were sacrificed at the onset of the disease. The remaining euglycaemic mice from each group were sacrificed at the end of the study and their pancreata specimens were collected for histological analysis of insulitis. Insulitis score is expressed as mean values ±SD

For statistical analysis each groups is compared to PBS-treated control mice:

b vs a, p=0.034 and; c vs a, p=0.002 by chi-square

e vs d, p=0.001 and f vs d, p=0.02 by one way ANOVA

### Example 9

Effect of PLB on Contractile Force of Rat Papillary Muscle

Background

In 1999, Johnson et al. demonstrated cardioprotective effects of PLB in 44 mongrel dogs. Shakarishvili et al. investigated the role of PLB in ischemic stroke using electron paramagnetic resonance (EPR) to quantify free radical production in the electron transport chain of mitochondrial membranes. Nicolletti, through western blot analysis, demonstrated lower levels of the cytokines TNF-α, Interferon-γ, IL-1, IL-12 and IL-18 in a dog model.

Results

The contractile force of rat papillary muscle bathed in 250 ml of oxygenated buffered solution was measured in rats. Optimal contractile force was obtained through progressive tissue lengthening (0.05 mm/5 minutes). One ampoule of PLB was administered at optimal contractile force and the derived contractile force was recorded.

Mutrie, et al. demonstrated a 38% increase in derived force (systolic force-diastolic force) of papillary muscle after administration of PLB (p=0.023, n=6). Six papillary muscles obtained from mice were studied in ex-vivo tissue baths.

Materials and Methods

The muscle ends were mounted to a force transducer (Harvard, Bioscience 529503) and a rigid hook to give isometric conditions inside a bathing chamber at 35.0-38.0° C. The initial equilibration period in low calcium control solution was approximately 20 minutes. The bath was then immersed in a high calcium control solution (high calcium control solution 1 L dH2O; 1.73 g NaHCO3, 0.277 g CaCl2, 0.2 ml insulin) and oxygenated with 95% 02-5% CO2. After adjustment of muscle length to give maximal isometric force, the muscles were stimulated on either side with supramaximal voltage. The tension recordings were analyzed for maximal twitch. The effects of PLB on the contractile parameters were evaluated and compared to those obtained during the initial equilibration period prior to PLB administration. The inhibitor-treated muscles were also compared to the control muscles at identical times. Statistical significance was assessed by a series of paired t-tests and p-values less than 0.05 were considered significant.

### References

9-1. Tbilisi State Medical University. Annals of Biomedical Research and Education. January 2002 (2):39.
9-2. Shakarishvili R, Sanikidze T, Mitagvaria N, Beridze M, Mikeladze D, Bakhutashvili V. The Role of Oxygen and Nitrogen Reactive Species in the Pathogensesis of Ischemic Stroke. Georgian State Medical Academy, Georgian Academy of Sciences (unpublished).
9-3. Rukhadze R, Sanikidze T, Bakhutashvili V, Chikovani T, Pantsulaia L, Jgenti M. Proceedings of the Georgian Academy of Sciences. 1998 (24):339-343.
9-4. Sharma R, Bolger A P, Li W, Davlouros P A, Volk H D, Poole-Wilson P A, Coats A J, Gatzoulis M A, Anker S. Elevated circulating levels of inflammatory cytokines and bacterial endotoxin in adults with congenital heart disease. American Journal of Cardiology. 92(2):188-93, 2003 Jul 15.
9-5. Heba G. Krzeminski T. Porc M. Grzyb J. Dembinska-Kiec A. Relation between expression of TNF-α, iNOS, VEGF mRNA and development of heart failure after experimental myocardial infarction in rats. Journal of Physiology & Pharmacology. 52(1):39-52, 2001 March.
9-6. Mariell J, Brozena S. Heart Failure. New England Journal of Medicine. 348 (20):2007-2018 May 2003.

### Example 10

Human DNA Sequence of Gene Encoding the ‘Parent’ Polypeptide of the Biologically Active Peptide in Plaferon-LB

Since the sequence of the peptide has been disclosed here, the nucleotide sequence capable of encoding this sequence can be deduced and the primer may be designed to “fish” for the gene which codes for the peptide or its precursor. This is the so-called “degenerated primer approach.” With a mixture of these degenerated primers, the nucleic acid molecules containing the sequence of the peptide capable of hybridizing the protein may be isolated and identified with human library. See, e.g., Molecular Cloning: A Laboratory Manual by Joseph Sambrook and David W. Russell.

The vector of the nucleic acid molecule encoding the sequence of the peptide can also be deduced using the sequence of the peptide disclosed herein. Vectors are well known in this filed. Said vectors could be plasmids. See e.g. Graupner, U.S. Pat. No. 6,337,208 entitled Cloning Vector, issued Jan. 8, 2002. See also Schumacher et al. U.S. Pat. No. 6,190,906 entitled Expression Vector fro the Regulatable Expression of Foreign Genes in Prokaryotes, issued Feb. 20, 2001.

Moreover, the cell containing the vector of the nucleic acid molecule encoding the peptide can also be deduced using the sequence of the peptide disclosed herein.

### Example 11

Comparison of the Peptide Composition of Two Batches of Plaferon-LB

Two milligrams of Plaferon-LB (PLB) (2 different batches) were dissolved in purified water and analyzed by RP-HPLC.

Chromatographic system: HP1100 with diode array detector (Agilent)

Chromatographic column: Alltech RP18, 5 μm

Buffer A: H20+TFA 0.1%

Buffer B: Acetonitrile+TFA 0.1%

Gradient: 0-100% B in 25 min.

Injection volumn: 100 μl

See FIG. 9 for the chromatographic profiles obtained. As illustrated in FIG. 9, concentrations of LAP are less, but the locations of the corresponding peaks are identical to the first batch. Concentrations are known to reflect minor differences in salt content between the two batches.

However, LAP is detected in both preparations with the same retention time and UV spectra proving the same amino acid sequence of LAP in both preparation of Plaferon-LB.

### Example 12

Peptide Composition of Plaferon-LB at Various Manufacturing Step

Two milligrams of Plaferon-LB (final product) and at two stages of manufacturing (Step I and II) were dissolved in purified water and analyzed by RP-HPLC as described in Example 11.

See FIG. 10 for the chromatographic profiles obtained. As illustrated in FIG. 10, LAP is present in each stage of manufacturing of Plaferon-LB. However, there is a smaller amount of LAP in the final product. The harsh conditions used for Plaferon-LB manufacturing may have partially broken down LAP.

### Example 13

Large-Scale Purification of LAP

Large-scale purification of LAP starting from a new batch of Plaferon-LB was performed.

Size Exclusion Chromatography

Thirty-five (35) vials of Plaferon-LB were dissolved in 3.5 ml of 0.9% NaCl. After dissolution, the compound contained in the Plaferon were separated in high MW (>5000 Da) and in low MW (<5000 Da) by size exclusion chromatography on Sephadex G25 medium (500 ml in an XK50/30 column, buffer: 10 mM ammonium bicarbonate pH 7.8 buffer, flow rate: 20 ml/min).

See FIG. 11 for the chromatographic profile obtained. One sample of both peak (low and high MW compounds) was analyzed by RP-HPLC (See FIG. 12). As expected, peak corresponding to LAP was found in the low molecular weight fraction. The peak containing low molecular weigh compounds (blue+black arrow) was pooled for further purification by RP chromatography.

Reverse Phase Chromatography

The fraction containing the low MW was further purified by RP chromatography on CG161

Matrix: CG161M (TosaHass)

20 ml in a HR 16/20 column

Sample: low molecular weight fraction of Plaferon-LB

Buffer A: water+0.1% TFA

Buffer B: acetonitrile+0.1% TFA

0-100% B in 87 min.

Flow rate: 9 ml/min

The peaks were manually collected and are currently freeze dried. FIG. 13 shows the chromatographic profile obtained.

### Example 14

Effects of LAP on Lipopolysaccharide (LPS)-Induced Septic Shock

Background

Intraperitoneal (i.p) or intravenous (i.v) injection with a single high dose (0.75-1.5 mg) of lipopoly-saccharide (LPS) extracted from the cell wall of Gram-negative bacteria provokes septic shock leading to lethality in 50-100% of mice within 3 days (See 14-1 to 14-4). This effect has been proven to be closely related to acute release into the bloodstream of Type 1 cytokines (IL-1, IL-2, TNF-α and IFN-γ and it is counteracted by Type 2 cytokines (IL-4 and IL-10) (See 14-1 to 14-4). The capacity of pharmacological compounds to reduce LPS-induced lethality is usually related to the inhibition of the production or the action of Type 1 cytokines, and/or to up-regulating the Type 2 cytokines (See 14-1 to 14-5). Murine LPS-induced lethality is therefore used as an in vivo tool to screen immunomodulatory compounds capable of down-regulating the synthesis/action of Type 1 cytokines or up-regulating Type 2 cytokines as well as to identify drugs with the potential to prevent and/or treat human endotoxemia (See 14-1 to 14-4).

In preliminary studies we have shown that the immunomodulatory peptide Lajor active peptide (LAP) exerts beneficial effects on the course of murine LPS-induced lethality (See Example 3 above). This study complements and extends our observation and evaluates the effects of LAP on LPS-induced increase in circulating cytokines. Mice treated with 10 microgram (mcg) LAP 1 h prior to and 1 after LPS exhibited a significantly lower rate of lethality than controls. In addition, mice so-treated had significantly lower blood levels of TNF-α, at 2 and 8 hours after LPS challenge. LPS-induced blood levels of IFN-γ and IL-10 were unaffected by LAP. Decreased lethality was noted when LAP was given only therapeutically, that is, only after the LPS challenge.

Materials and Methods

Animals

Six week old female CD1 mice (Charles River, Calco, Italy) were kept under standard laboratory (non specific pathogen free) with free access to food and water.

Induction of Septic Shock and Experimental Treatment

To induce lethal endotoxemia, the mice were injected i.p. with 1 mg lipopolysaccharide (LPS, Cod. L6011, lot 112K4063, Sigma Chimica, Milan, Italy). Six groups of mice were created, treated according to the experimental design shown in the Table.ip. LAP was provided by Lajor BioTech (Pittsburgh, Pa. USA), dissolved volume/volume in trifluoroacetic acid 0.1% in water and Na2HPO4 and injected ip in a final volume of 100 mcl.

Effects of LAP Treatment on LPS-Induced Blood Levels of TNF-α, IFN-γ and IL-10

To evaluate the impact of LAP-treatment on the increase in cytokines by LPS in the circulation of the mice, experiments were carried out where mice treated with 10 mcg LAP or vehicle as described were sacrificed just before injection of a sublethal (0.5 mg/mouse) dose of LPS (T0, hence this group of mice received only one treatment with LAP) and then 2 and 8 hours after LPS (n=10 mice at each time point). Plasma samples were obtained by blood obtained from individual mice at sacrifice. TNF-α, IFN-γ and IL-10 were measured by mouse specific solid-phase ELISA according to the manufacturer's (Celbio Euroclone, Milan, Italy) instructions. Intra and inter-assays coefficient of variations were within 10%. The limit of sensitivity of the assays were 7 pg/ml. For statistical analysis, samples with undetectable amounts of cytokine were assigned 7 pg as theoretical value.

Statistical Analysis

Statistical analysis was performed by chi-square for lethality and one way ANOVA for cytokine measurements. P values lower than 0.05 were taken as significant.

Results

LAP Prophylaxis Markedly Reduces LPS-Induced Lethality

As expected most of the vehicle-treated control mice died within 72 hours after injection of LPS. The mice treated with 1 or 20 mcg LAP exhibited kinetic and cumulative rate of lethality very similar to that of control mice regardless of the administration regime. In contrast, the mice treated with 10 mcg LAP exhibited a dramatic reduction of lethality. This dose of LAP was equally effective whether it was administered −24 and −1 h prior to LPS or 1 hour prior to and 1 hour after LPS (see Table 7 and FIG. 14). LAP did not elicit a detectible effect however when administered as a “therapeutic” one hour after LPS injection. (See Table 8 and FIG. 14)

TABLE 8
Experimental design: time and dose effects
of LAP on LPS-induced lethality

| | Time of | | | Lethality | | |
|---|---|---|---|---|---|---|
| Treatment | administration | Dose | | 72 h | | P |
| Vehicle | −24, −1 h | 0.1 | ml | 12/16 | (75%) | Control |
| (n = 16) | | | | | | |
| LAP (n = 14) | −24, −1 h | 1 | mcg | 11/14 | (79%) | N.S. |
| LAP (n = 14) | −24, −1 h | 10 | mcg | 2/14 | (14%) | P = 0.003 |
| LAP (n = 14) | −1, +1 h | 10 | mcg | 2/14 | (14%) | P = 0.003 |
| LAP (n = 14) | −1, +1 h | 20 | mcg | 14/14 | (100%) | N.S. |
| LAP (n = 14) | +1 h | 10 | mcg | 10/14 | (71%) | N.S. |

For statistical analysis each group is compared to vehicle-treated controls. Statistical analysis was performed by chi-square.

LAP Suppresses LPS-Induced Increase in Circulating Levels of TNF-α

Injection of LPS is associated with a marked increase in the blood levels of both type 1 (IFN-γ, TNF-α, IL-1) and type 2 (IL-10) cytokines that occurs with different kinetic after the inoculation of the toxin. To evaluate the effects of LAP treatment on LPS-induced cytokine increase in the circulation of the mice, experiments were carried out where mice treated with 10 mcg LAP or its vehicle, were sacrificed just before injection of a sublethal dose of LPS one hour after treatment with either LAP or PBS (T0) and then 2 (T2) and 8 (T8) hours after LPS.

When sacrificed at T0 just before of the injection of LPS none of the control mice had detectable amounts of IFN-γ, TNF-α and IL-10 in the circulation (see FIG. 15A-C). Although neither IFN-γ nor TNF-α could be detected in the circulation of mice treated with LAP, we observed that 3 out of 10 mice receiving LAP 1 hour before sacrifice had detectable levels of IL-10 in the blood (See FIG. 15A-C).

At 2 and 8 hours after injection of LPS a characteristic modification of circulating levels of these cytokines was observed in control mice characterized by an early increase (T2) of TNF-α and IL-10 followed by a later (T8) increase of IFN-γ (see FIG. 15A-C). Relative to these control mice, the mice treated with LAP exhibited significantly lower blood levels of TNF-α both at 2 (37.3% reduction vs. controls, p=0.01) and 8 (76.5% reduction vs. controls, p=0.005) hours after LPS (see FIG. 15A-C). In contrast LAP-treatment did not modify the blood levels of IFN-γ or IL-10 (See FIG. 15A-C)

Conclusions

The present results indicate that when administered as a prophylactic, that is prior to administration of the toxin, at the dose of 10 mcg, LAP powerfully counteracted the lethal effects of a high dose of LPS in mice. We also observed that mice treated with LAP had significantly lower amounts of TNF-α than the vehicle-treated-control group. In contrast, there were no significant differences in either LPS-induced IL-10 or IFN-γ blood levels between LAP-treated and vehicle-treated mice. We have however noticed that 1 hour after treatment with 10 mcg LAP 3 out of 10 mice had detectable blood levels of IL-10 compared to 0 out of 10 controls.

Because endogenous TNF-α has been repeatedly proven to play a major pathogenic role in murine LPS-induced lethality (See 14-5) it seems likely that reducing LPS-induced TNF-α synthesis might have been causally related to the beneficial effects of LAP in this model.

Inhibition of TNF-α synthesis may represent an important immunopharmacological mode of action of LAP. In fact, TNF-α has been conclusively demonstrated to play a major pathogenic role in several immuno-inflammatory and auto-immune diseases in humans including rheumatoid arthritis, Crohn's disease, psoriasis and inflammatory dermatoses (6-8). Hence, the antagonistic action of LAP on TNF-α synthesis may be an important application for this peptide for the treatment of these and possibly other TNF-α mediated immunopathological conditions.

The main outcome of this study to be the clear-cut demonstration of clinical (reduction of lethality) and immunopharmacological (reduction of LPS-induced increase in TNFα blood levels) activity achieved by LAP prophylaxis in an aggressive model of acute immunoinflammation such as LPS-induced lethality. This provides strong proof of concept for the potential utility of LAP in other immuno-inflammatory or auto-immune diseases where TNF-α and possibly other type 1 cytokines play a major pathogenetic role.

### References

14-1. Nicoletti F., et al. Prevention and treatment of lethal murine endotoxemia by the novel immunomodulatory agent MFP-14. Antimicrob. Agents Chemother, 45: 1591, 2001
14-2. Genovese F., et al. Antimicrobial Agents and Chemotherapy, 40: 1733, 1996
14-3. Nicoletti F. ,et al. Prevention of endotoxin-induced lethality in neonatal mice by interleukin-13. Eur. J. Immunol., 27: 1580, 1997
14-4. Nicoletti F., et al. Endotoxin-induced lethality in neonatal mice is counteracted by interleukin-10 (IL-10) and exacerbated by anti-IL-10. Clin. Diagn. Lab. Immunol., 4: 607, 1997
14-5. Scallon B J, et al. Functional comparisons of different tumour necrosis factor receptor/IgG fusion proteins. Cytokine, 7: 759, 1995
14-6. Nahar I K, Shojaunia K, Marra C A, Alamgir A H and Anis A H. Infliximab treatment of rheumatoid arthritis and Crohn's disease. Infliximab treatment of rheumatoid arthritis and Crohn's disease. Ann. Pharmacother. 37:1256, 2003
14-7. Victor F C and Gottlieb A B. TNF-α and apoptosis: implications for the pathogenesis and treatment of psoriasis: J. Drugs Dermatol., 1: 264, 2002
14-8. Drosou A., et al. Use of infliximab, an anti-tumor necrosis factor alpha antibody, for inflammatory dermatoses. J. Cutan. Med. Surg., 7: 382-386, 2003
14-9. Li M C and He S H. IL-10 and its related cytokines for treatment of inflammatory disease. World J. Gastroenterol., 10: 620, 2004

### Example 15

The Effects of LAP in Murine Concanavalin A-Induced T Cell-Dependent Immuoinflammatory Hepatitis

Background

Con A-induced hepatitis is a cell-mediated immuno-inflammatory condition similar to human auto-immune hepatitis that can be induced in mice by a single intravenous (iv) injection of Concanavalin (Con) A (See 15-1 to 15-9). This disease is characterized by a marked increase in the plasma levels of transaminase shortly (8-24 hours) after Con A challenge and simultaneous infiltration of the liver with neutrophils, macrophages and T cells followed by apoptosis and necrosis of the hepatocytes (See 15-1 to 15-9). It has been proposed that Con A injection provokes the migration of splenic T cells to the liver where they damage hepatocytes through release of perforin/granzymes and activation of macrophages (See 15-4). The contribution of T cells in this model is underscored by the resistance of nude athymic mice to the hepatitis-inducing effects of Con A and by the preventive effects of drugs targeting T cells, for example cyclosporin A, FK506 and sodium fusidate (See 15-1, 15-2, 15-5). The use of exogenously administered cytokines and specific cytokine antagonists along with studies in genetically engineered mice have clearly demonstrated that each of the cytokines IL-4, IFN-γ (and TNF-α is essential for development of the disease, while IL-6 and IL-10 downregulate the immunoinflammatory attack on the liver cells (See 15-1 to 15-3, 15-6 to 15-9).

Lajor active peptide (LAP, Lajor Biotech, Pittsburgh, USA) is a peptide endowed with immunomodulatory properties that we have previously shown to be capable of counteracting murine lypopolisaccharide (LPS) induced lethality in mice. Because this latter model is known to be dependent on TNF-α and since treatment with LAP significantly reduced the LPS-induced increase in TNF-α blood levels, these observations prompted us to test the effect of LAP prophylaxis on the development of murine Con A-induced hepatitis.

The results show that the marked increase in transaminases provoked in PBS-treated control mice within 8 hours after Con A-challenge was powerfully reduced by a short prophylactic treatment with LAP.

Materials and Methods

Animals

Eight weeks old outbred CD1 male mice (Charles River, Calco, Italy) were kept under standard laboratory conditions (non-specific pathogen free) at 24° C. with free access to food and water. The food was withdrawn 16 hours prior to the experiments.

Hepatitis Induction

Con A (Sigma Chemical, St. Louis, Mo.), dissolved in sterile phosphate-buffered saline (PBS) was injected into the tail veins. The groups were treated with either LAP (dissolved volume/volume in trifluoroacetic acid 0.1% in water and Na2HPO4 and then further diluted in water for injection), or its vehicle, 1 hour prior to and one hour after Con A. An additional group of control mice was injected with Con A and received no treatment. Finally, other two groups of mice were also included for comparison that were either injected i.v. with PBS or received no treatment (Table 8). LAP, its vehicle, PBS and Con A were all injected in a final volume of 100 microliter (mcl). The animals were sacrificed for blood collection 8 hours after Con A injection, when biochemical and signs (transaminases increase) of hepatic injury are pronounced (15-1 to 15-9). Mice dead before sacrifice (Table 8) were not included.

Assay for Transaminase Activity

Plasma alanine aminotransferase (ALAT) activity was determined by a standard photometric assay using a bichromatic analyzer. Results are expressed in U/L

Statistical Analysis

Results are shown as mean values ±SD. Statistical analysis was performed by one way ANOVA. The effect of LAP was considered to be statistically significant when the difference of ALAT blood levels versus controls yields a p value at least lower than 0.05.

Results

Powerful Reduction of Con A-Induced ALAT Increased by LAP Prophylaxis

As expected, 8 hours after the iv injection of PBS the blood values of ALAT were very similar to those of unchallenged normal mice (See Table 9 and FIG. 15). In contrast, a marked increase in the blood levels of ALAT was observed in vehicle-treated control mice within 8 hours after challenge with Con A (See Table 9 and FIG. 15). This increase was significantly reduced by a short prophylactic course with LAP, mice so-treated exhibiting significantly lower values of ALAT than controls 8 hours after Con A (84.7% reduction) (See Table 9 and FIG. 15).

TABLE 9
Reduction of Con A-induced ALAT by LAP

| | | | ALAT | |
|---|---|---|---|---|
| Treatment | Con A | PBS | values | Lethality |
| Nil (n = 15) | + | − | 4534 ± 831a | 2/15 |
| Vehicle (n = 15) | + | − | 4227 ± 693 | 3/15 |
| LAP (n = 15) | + | − | 686 ± 197b | 1/15 |
| Nil (n = 15) | − | + | 34 ± 12 | 0/15 |
| Nil (n = 10) | − | − | 28 ± 10 | 0/15 |

Discussion

We have shown here that a short prophylactic treatment with LAP causes a significant reduction in ALAT blood levels compared to vehicle-treated control mice. Because the increase in transaminase values in this model is known to be closely related to histological signs of liver damage provoked by infiltrating T lymphocytes, macrophages and neutrophils (See 15-1 to 15-5), the present results are strongly suggestive for a powerful preventive efficacy of LAP prophylaxis on the development of serological and also histological signs of Con A-induced hepatitis.

The present finding extends to this model of acute cell mediated immuno-inflammation the beneficial anti-inflammatory effect observed with LAP in LPS-induced lethality in mice. In addition, the apparent capacity of LAP to inhibit TNF-α synthesis in the latter model along with the central pathogenic role of this cytokine in Con A-induced hepatitis (See 15-3) suggests that antagonizing TNF-α production might have also been involved in the anti-hepatitic effects of LAP. The presently demonstrated prophylactic capacity of LAP could have important implications for the clinical use. LAP could for example be administered to patients with auto-immune hepatitis during spontaneous and/or pharmacological-induced remission periods of the disease so to prevent re-exacerbations and it could also be used to prevent immuno-inflammatory liver events that can follow hepatitis B viral infection and that can contribute to chronicization of the disease and development of cirrhosis.

### References

15-1. Tiegs G. J. et al. A T cell-dependent experimental liver injury in mice inducible by concanavalin A. J. Clin. Invest., 90: 196, 1992
15-2. Mizuhara H., et al., T-cell activation-associated hepatic injury : mediation by tumor necrosis factor and protection by interleukin-6. J. Exp. Med., 179: 1529, 1994
15-3. Gantner F., et al. Concanavalin A-induced T-cell-mediated hepatic injury in mice: the role of tumor necrosis factor. Hepatology, 21: 190: 1995
15-4. Watanabe Y. et al. Concanavalin A induces perforin-mediated but not Fas-mediated hepatic injury. Hepatology 24: 702, 1996
15-5. Nicoletti F., Beltrami B., Raschi E., Di Marco R., Magro G., Grasso S., Bendtzen K., Fiorelli G., Meroni P L. Protection from concanavalin A (ConA)-induced T cell-dependent hepatic lesions and modulation of cytokine release in mice by sodium fusidate. Clin. Exp. Immunol., 110: 479-484 , 1997

15-6. Xiang M., Zaccone P., Di Marco R., Magro S., Di Mauro M., Beltrami B., Meroni P L., and Nicoletti F. Prevention by rolipram of concanavalin A-induced T-cell dependent hepatitis in mice. Eur. J. Pharmacol., 367: 399-404, 1999
15-7. Di Marco R., Xiang M., Zaccone P., Leonardi C., Franco S., Meroni P L., and Nicoletti F. Concanavalin A-induced hepatitis in mice is prevented by Interleukin (IL)-10 and exacerbated by endogenous IL-10 deficiency. Autoimmunity, 31 : 75-83, 1999
15-8. Nicoletti F., et al., Essential pathogenetic role for intereferon (IFN)-γ in Concanavalin A-induced T cell dependent hepatitis: Exacerbation by exogenous IFN-γ and prevention by IFN-γ receptor Immunoglobulin fusion protein. Cytokine, 12 : 315-323, 2000
15-9. Nicoletti F., Di Marco R., Zaccone P., Salvaggio A., Magro G., Bendtzen K., and Meroni P L. Murine concanavalin A-induced hepatitis is prevented by interleukin (IL)-12 antibody and exacerbated by exogenous IL-12 through an interferon-γ-dependent mechanism. Hepatology, 32 : 728-733, 2000

### Example 16

Preliminary Report on the Effects of Lajor Active Peptide (LAP) in the Development of Auto-Immune Diabetes in the Non-Obese Diabetic (NOD) Mouse

Materials and Methods

Animals

Female NOD mice (Charles River, Calco, Italy) were maintained under standard laboratory conditions (non-specific pathogen free) with free access to food and water. During the study period of diabetes prevention the mice were screened for diabetes development twice a week by means of glycosuria followed, when positive, by measurement of glycaemia. Mice are diagnosed as diabetic when fasting glycaemia is above 12 mmol/l for 2 consecutive days.

Experimental Treatment

Euglycaemic female NOD mice were randomly allocated into 4 different groups receiving either LAP or vehicle starting at the 4^{th} or at the 12^{th} week of age. Because insulitis is virtually absent in 4-week-old NOD mice and is actively ongoing at 12 weeks, this approach allowed us to investigate the effects of LAP-treatment in both the early and late diabetogenic stages of the NOD mouse. The readout of the early prohylactic treatment was to evaluate the effect of LAP on development of insulitis, while the readout of the late prophylactic treatment was to evaluate the effect of LAP on the incidence of clinically overt diabetes.

Early Prophylaxis

For the early prophylactic treatment 4-week-old NOD mice were treated with either 10 mcg LAP (dissolved and diluted [100 mcl final volume] as described in Example 14 and 15) or 100 mcl vehicle, daily, six times weekly until the age of 14 weeks. At this point, the euglycaemic mice were sacrificed and their pancreatic specimens collected for histological examination of insulitis.

Late Prophylaxis

For the late prophylactic treatment, 12-week-old NOD mice were randomly divided into two experimental groups, one treated with LAP and the other with vehicle, under the same experimental regime described for the early prophylactic treatment. Treatment will be continued.

Histological Examination of Pancreatic Islets

Histological examination of the pancreatic islets was performed in a blind fashion by two pathologists unaware of the status and/or the treatment of the animals, as described previously (See 16-3 to 16-5). The degree of mononuclear cell infiltration is graded as follows: 0, no infiltrate; 1, peri-ductular infiltrate; 2, peri-islet infiltrate; 3, intra-islet infiltrate; 4, intra-islet infiltrate associated with β-cell destruction. At least 12 islets are counted for each mouse. The mean score for each pancreas is calculated by dividing the total score by the numbers of islets.

Results

Lack of Toxicity of LAP

Long-term administration of LAP to NOD mice either from the age of 4 to 14 weeks or from 12 to the 25^{th} weeks of age was apparently well tolerated by the animals as judged both from their clinical appearance and behaviours. In addition, the body weight of LAP-treated animals was similar to that of control animals throughout the study period, and no differences were observed in both azotemia and transaminases values at the end of the study in the mice sacrificed at week 14^{th} for the histological analyses (not shown). FIG. 17A shows the lack of effect of prolonged treatment (14-25 weeks) with LAP on body weight gain in NOD mice.

Early Prophylactic Treatment with LAP Reduces the Severity of Insulitis in NOD Mice

Two out of 8 (25%) NOD mice treated with vehicle from the 4^{th} week of age developed diabetes before the end of the study at the age of 14 weeks and were therefore sacrificed and not included for histological analyses. None of the NOD mice treated with LAP developed diabetes during the treatment period. In agreement with this apparent clinical beneficial effect of early prophylactic treatment with LAP, the insulitis score of the LAP-treated mice was found to be significantly lower than that of control mice treated with vehicle (0.9±0.6 vs 2±0.9, p00.018)(See FIG. 17B).

Discussion

We have shown here that when administered upon early prophylactic regime to 4 week old NOD mice for 10 consecutive weeks LAP significantly reduced the severity of the insulitis process in these animals. That this histological effect might have clinical efficacy is suggested by the fact that none of the mice treated with LAP developed diabetes until age 14 weeks compared to 2 of 8 controls.

### References

16-1. Rabinovitch A. An update on cytokines in the pathogensis of insulin-dependent diabetes mellitus. Diabetes Metab. Rev., 14: 129, 1998
16-2. Bach J F. Immunotherapy of type 1 diabetes: lessons for other auto-immune diseases. Arthritis Rheum., 4 Suppl 3: S3-15, 2002
16-3. Nicoletti F et al. Fusidic acid and insulin-dependent diabetes mellitus. Autoimmunity 24:187, 1996
16-4. Nicoletti F et al The effects of a nonimmunogenic form of murine soluble interferon-g receptor on the development of auto-immune diabetes in the NOD mouse. Endocrinology, 137:5567-5575, 1996
16-5. Nicoletti F et al. Early prophylaxis with recombinant human Interleukin-11 prevents spontaneous diabetes in NOD mice. Diabetes, 48: 2333-2339, 1999

### Example 17

Use of PLB in the Treatment of Leishmaniasis

It has been found that PLB seems to exert beneficial effects in the treatment of leishmaniasis in a subject. In an embodiment, the subject is an animal.

Materials and Methods

Subcutaneous administration of PLB to a group of 10 dogs with manifest clinical symptoms of leishmaniasis (peripheral lymphadenopathy and skin lesions of a high degree, mainly represented by sores and bleeding ulcers with loss of substance, anorexia and weight loss), at the doses and times indicated in Table 10, led to substantial reduction of the symptoms.

Results and Conclusions

No adverse effects were observed during the treatment. These findings demonstrate that the administration of PLB cures the clinical symptoms of leishmaniasis in a totally safe manner.

The compositions in the form of solutions or suspensions in the preferred aqueous sterile solvents of 10 ml were administered to subjects suffering from leishmaniasis by the parenteral route, in particular subcutaneously or intramuscularly, until the disappearance or substantial reduction of the symptoms.

TABLE 10
Dosages and dosage application of PLB to dogs
with clinical symptoms of leishmaniasis

| | |
|---|---|
| Patient's weight < 10 kg | 1 phial/day subcutaneously for 6 days |
| | 7^{th} day: rest |
| | 1 phial/day subcutaneously for 6 more days |
| Patient's weight > 10 kg | As above, but doubling the dose: 2 phial/day |

### Example 18

Cosmetic Uses of PLB

To determine anti-wrinkle properties of PLB, a total of 200 women were given PLB-lanoloin based ointment for 14 days to be applied on right half of the face, with left side of the face serving as a control. Substantial improvement was reported by participating cosmetologists. All 200 women used PLB-lanoloin based ointment on left side (controls) of face to even out the results.

A moisturizing creme and a more concentrated “serum” containing PLB or the synthetic form of the peptide or polypeptide of the present invention with a cosmetic carrier or an additive, such as additive (A) or (B) as described below, were tested on at least 50 people in the United States, Europe and Asia. The subjects have all types of complexions, wrinkles, bug bites (allergic reactions like bee stings and poison ivy), psoriasis, first or second degree skin burns, trauma, exposure to the sun and UV, shingles rash (herpes zoster), and/or rashes associated with Lupus Erythematosis, diabetic ulcers, skin grafts. The creme or serum improved the appearance and condition of the skin which have been damaged from almost any cause. Moreover, gray or white hairs which have been treated with the serum have been reported to regain its original color after treatment.

Additive (A): Purified Water, Glyceryl Stearate (and) Laureth 23, Glycerine, Acetylated Monoglyceride, Coconut Oil, Aloe Barbadenis Leaf Juice, Safflower Oil, Stearic Acid, Oleic Acid, Cetyl Alcohol, Mineral Oil, Lanolin, Laneth 16, Tocopherol Acetate (Vitamin E), Propylene Glycol (and) Methylparaben (and) Propylparaben (and) Diazolidinyl Urea, Jojoba Oil, Carbomer, PLB, Retinyl Palmitate (Vitamin A), Triethanolamine, Fragrance, BHT.

Additive (B): Purified Water, PLB, Polyacrylamide C13-C14 (and) Isoparaffin (and) Laureth 7, Propylene Glycol, Isopropyl Alcohol, Glycerin, Dimethicone, Potassium Hydroxide, Diazolidinyl Urea, Iodopropynyl Butylcarbamate, Fragrance.

### Example 19

Mechanism of Action

Possible mechanism of action include specific inhibition of cathepsin S thereby reducing the competency of class II MHC molecules for binding antigenic peptides, reducing presentation of antigenic peptides by class II MHC molecules and suppressing immune response, modulation of apoptosis by dose-dependant reduction or increase of TNFα, restoring the impaired electron transport in mitochondrial respiratory chain and anti-inflammatory action exerted by inhibition of phosholipase A₂.

### Example 20

PLB Anti-Teratogenic Activity Study

The mechanism of teratogenic effect caused by Cyclophosphamide (CP) includes activation of apoptosis. Influence of Plaferon LB on intensity of apoptosis was studied in brains of fetuses from mice treated with CP by TUNEL method.

There were three (3) groups of animals:

Group (A): no treatment; controls (12 animals). Group (B): treated with CP only (18). Group (C): treated with CP and Plaferon LB (18).

CP (15 mg/kg) was injected to pregnant mice of groups B and C intraperitoneally at 12^{th} day of gestation. Plaferon LB (0.8 mg/kg) was introduced to group C by the same rout 3 times−1 hour prior to CP injection, then after 3 and 6 hours. Animals were euthanized at 18^{th} day of gestation, their fetuses were collected and studied.

It was found that treatment with Plaferon LB lowered the ratio of apoptotic cells in brains of fetal mice compared to group of animals treated with CP only and provided anti-teratogenic effect.

5-6 μM sections of murine brain from all 3 groups of fetuses stained by TUNEL method. Dark spots represent apoptosis. See FIG. 19(A)-(C). (A) Control (no treatment). (B) CP only. (C) CP +Plaferon LB.

Fetuses from group A had no deformities, group B had 64.8% deformities and group C had only 11.2% deformities.

Fetus from B group of animals (CP only) presented typical deformities, i.e., ectrodactily syndrome (anomaly of limbs), cleft pallet, kinked tail and low body mass. See FIG. 19D.

Fetus from C group of animals treated with CP and PLB shows no deformity and appears to have normal weight/size. See FIG. 19E.

### Example 21

Effects of LAP on T Cell Proliferation

T cell proliferation assays were set up as follows. Antigen presenting cells (APCs) obtained as single cell suspension from Balb/c mouse spleens were pulsed with fecal extract (from Balb/C mice) overnight in 24 well-plate (1×10⁶ cells/ml with 400 μg/ml of extract). Responding cells were obtained from Balb/c mouse spleens as CD4⁺CD25⁻ T cells. CD4+ T cells were selected by DYNABEADS, and CD4⁺CD25⁻ T cells were then selected using labelled anti-CD25 antibody (e.g. PE-labelled anti-CD25). Responding T cells were co-cultured with APC in 96 round bottom well-plate (3.3×10⁴ responding cell/1×10⁵ APC in 200 μl) in the presence or absence of LAP for 5 days. Immune cell proliferation was then determined by standard thymidine incorporation assays.

CD4⁺CD25⁻ T cell proliferation was inhibited in the presence of LAP (FIG. 20), whereas T cell proliferation was not affected in response to APCs pre-treated with LAP for 24 hours (FIG. 21). T cell proliferation in the presence of control peptide or T cell proliferation in response to APCs pre-treated with control peptide was not affected (FIG. 22), indicating that the inhibition of T cell proliferation is specific to LAP. Taken together, since CD4⁺CD25⁻ T cells are naïve or non-activated T cells, these data indicate that LAP is capable of regulating the proliferation and maturation of T cells.

### REFERENCES

1. Bakhutashvili A, Chikovani T, Bakhutashvili V, Imedidze E. Immunopharmacology of preparation Plaferon-LB. Intern J Immunorehab 1994; 1 (S): 44.
2. BakhutasBhvili A, Cheishvili N, Chikovani T, Bakhutashvili V. Plaferon LB—a new immunodilatory drug. Abstracts XVI European Congress of Allergology and Clinical Immunology, Madrid, Spain, 25-30 Jun. 1995. Europ J Allergy Clin Immunol 1995; 50(26): 9. Abstract # OC-009.
3. Bakhutashvili A, Jaguzhinsky L, Bakhutashvili I, Kadagidze Z, et al. Amnion apoptosis modulator. Int J Immunorehab 2001, 3(2): 17-22.
4. Bakhutashvili V, Bakhutashvili A, Chikovani T, Cheishvili N. Immunomodilatory activity of Plaferon-LB. Georg Symp Project Development and Conversion; Collection of Reports 1995; 189-191.
5. Bakhutasvili V, Chikovani T, Rukhadze R. The perspectives of a new immunomodulator Plaferon LB in endocrynologic practice. Proceedings of Tbilisi State Medical University 1996; 32: 21-23.
6. Bakhutashvili V, Malashkhia V, Mikeladze D, Chikhladze M, Malashkhia Y, Bakhutashvili A. Impact of Plaferon-LB upon drug-resistant forms of epilepsy. Int J Immunorehab 1996; 3: 28-37.
7. Bakhutashvili V, Javakhishvili N, Tsagareli Z, Kipshidze N. Cardioprotective effects of Plaferon LB in a canine model. The J Heart Failure 1997 May; 4(1): 38. Abstract #151.
8. Bakhutashvili V, Gagua M, Garishvili T, Gelashvili L, Kharebava G, Kvaratskhelia E, Menteshashvili A. Human placenta antioxidant compounds of peptide nature. 11th International Symposium on Atherosclerosis, Paris 1997 October 5-9, Int J Res Invest on Atherosclerosis and Related Diseases October 1997; 134(1-2): 199. Poster # 3. P. 4.
9. Bakhutashvili V, Chikovani T, Bakhutashvili I, Cheishvili N, Kukuladze N, Bakhutashvili A. Some pharmacological characteristics of Immunomodulator Plaferon-LB. 10th International Congress of Immunology in New Delhi, 1998 Nov. 1-6; The Immunologist 1998 Suppl 1: 586.
10. Bakhutashvili V, Shakarishvili R, Geladze T, Tatishvili N, Bakhutashvili A, Cheishvili N, Chikovani T, Sobera L A. Drugs of the Future 1999; 24(9): 974-977.
11. Bakhutashvili V, Telia D, Katsarava V. Use of Plaferon-LB and cromoglycate in patients with symptomatic asthma. Int J Immunorehab 1999; 11: 214-215.
12. Bakhutashvili V, Pagava K, Telia A, Jorjoladze N, Kokaia L, Alavidze M, Kvachadze L. Bronchotropic effect of Plaferon-LB. Int J Immunorehabilitation 1999; 5: 51-53.
13. Bakhutashvili V, Chikovani T, Bakhutashvili A, Dolidze T, Nanava N, Kvitaishvili G. Dynamics of some immunological indices in case of Plaferon-LB therapy. Europ. J. Allergy and Clin Immun 1999; 54(52 S): 77. Abstract # P37.
14. Bakhutashvili V, Chikovani T, Bochorishvili T, Metreveli D, Pavliashvili D. Results of treatment of acute B hepatitis by Plaferon LB. Bulletin of Georgian Academy of Science, Biology Series 1999 25 (1-3): 21-23.
15. Bakhutashvili V, Bakradze I, Aladashvili A. Impact of perioperational immunotherapy upon nonspecific resistance and specific immune status in patients with bowel cancer and upon frequency and spectrum of complications in nearest postoperative period. Int J Immunorehab 2000; 2 (2): 4. Abstract # 3.
16. Beridze M. Effect of Plaferon-LB on several clinical and neuroimmunological indices. Georgian Medical News 1998; 6(39): 27-30.
17. Beridze M, Malashkhia V, Bakhutashvili V. Neuroprotective action of immunomodulator Plaferon-LB. Int J of Immunorehab 1999; 14: 114. Abstract # 15.
18. Chavchanidze D, Hvadagiani G, Kalmahelidze V, Sulhanishvili V, Stepina J, Bakhutashvili V, Managadze L. Protective effect of Plaferon LB preparation on acute ischemic renal injury in experiments. Archivum Urologium Belgrade 1989; 30: 45-51.
19. Chavchanidze D, Sanikidze T, Sulkhanishvili V, Bakhutashvili V, Managadze L. Changes of blood paramagnetic centers under the influence of shock waves on kidneys and membrane-protector effect of Plaferon-LB in experiment. Bulletin of the Georgian Academy of Sciences 1998; 158(2): 332-335.
20. Chavchanidze D, Sanikidze T, Bakhutashvili V, Managadze L. Determination of traumatic influence of shock waves and membrane-protecting effects of Plaferon-LB on the renal parenchyma during extracorporeal lithotripsy in experiment. Proc Georgian Acad Sci; Biol Ser 1998; 24(1-6): 53-59.
21. Cheishvili N, Kukuladze N, Bakhutashvili A, Bakhutashvili V. Effect of Plaferon-LB on proliferative activity of human periferal blood mononuclear cells and murine splenocytes. Reports of Georgian Academy of Science 1994; 150 (1): 142-143. Ch
22. Chichua G, Sanikidze T, Bakhutashvili V. Correction of the induction of nitric oxide synthesis and free radical reactions in experimental model of vitro-retinopathy by Plaferon-LB. Aller & Immunol 2 (1): 155-161.
23. Chikovani T, Cheishvili N, Bakhutashvili A, Bakhutashvili V. The influence of Plaferon LB on synthesis of interleukins. Int J Immunorehab 1996; 3: 96
24. Chikovani T, Cheishvili N, Pirtskalava T, Pantsulaia I, Bakhutashvili V. Plaferon LB as a blocker of neurotoxic effect of glutamate. Int. J Immunorehab 1997; 5: 46.
25. Chikovani T, Cheishvili N, Pantsulaia I, Bakhutashvili V. Immunopotential activity of Plaferon LB fractions. Int J Immunorehab 1998; (9): 5.
26. Chikovani T, Pavliashvili D, Bakhutashvili V. Different ways of therapy by Plaferon LB in patients with acute B viral hepatitis. Abstracts of International Falk Workshop, New Aspects in Hepatology and Gastroenterology; May 29-30, 1998; Tbilisi, Georgia. Abstract # 52.
27. Chikovani T, Cheishvili N, Pantsulaia I, Bakhutashvili V, Bakhutashvili A. Immunologic study of human amniotic factor. Eur J Allergy Clin Immun; 1999; 54 (52 S): 89. Abstract # P81.
28. Chikovani T, Rukhadze R, Bakhutashvili V, Sanikidze T, Pantsulaia I. Antioxidant action of immunomodulatory drug Plaferon LB in experimental thyroid pathology. Int J Immunorehab 1999; 12(S): 14-18.
29. Dolidze C, Gelovani M, Bakhutashvili V, Chikovani T. Antirelapse effect in case of idiopatic nephropathy syndrome in children. Bull Acad Sci Geo 1992; 145 (1): 209-11.
30. Gagua M, Russia L, Kupatadze R, Simonidze M, Bakhutashvili V. Investigation of polipeptide contents of Plaferon LB. Bulletin of Acad of Sci of Georgia 1996; 3(153): 450-452.
31. Gagua M, Dzidziguri D, Bakhutashvili V. Influence of Plaferon LB on the transcriptional activity of regenerating liver and kidney cells. International-European A.I.R.R. Conference; Tbilisi, 1999 Oct. 4-9; Collection of reports: 11.
32. Gagua M, Dzidziguri D, Mikadze E, Rukhadze M, Bakhutashvili I, Bakhutashvili V. Effect of Plaferon LB on morpho-functional activity of hepatocytes under conditions of hormonal disbalance in white rats. Bulletin of Georgian Acad of Sci 1999; 160 (3): 56-68.
33. Germanashvili T, Pavliashvili N, Sanikidze T, Bakhutashvili V. Thymic paramagnetic centers changes during crush syndrome of limbs and its correction with Plaferon-LB. Exper & Clin Medicine 2000; 1: 30-33.
34. Gongadze M, Chicovani T, Sanikidze T, Bakhutashvili V. Effect of Plaferon-LB on LPS-induced synthesis of nitric oxide. Int J Immunorehab 2001; 3 (3): 158-159.
35. Gongadze M, Chicovani T, Sanikidze T, Bakhutashvili V. Correction of LPS-induced synthesis of nitric oxide. Bulletin of Georgian Acad of Sci 2002; 166 (2): 311-313.
36. Gurgenidze G, Chogovadze M, Bakhutashvili V. Plaferon-induced in vitro inhibition of mitogen-activated T-lymphocyte proliferation in steroid-resistant asthmatic patients. Trans-Caucasian J Immunology 1999; 1 (2): 12-19.
37. Johnson D W, Kipshidze N, Javahishvili N, Zagareli Z, Bakhutashvili V. Cardioprotective effects of Plaferon-LB in canine model. First International Congress on Heart Disease-New Trends in Research, Diagnosis and Treatment;

Washington D.C., USA; 1999 May 16-19; Book of Abstracts: 46. Abstract # 30.
38. Macharadze D, Bakhutashvili V, Fedotova E. Plaferon-LB in immuno-rehabilitation of chidren with bronchial asthma. Int J Immunorehab 1998; 8: 67. Abstract # 249.
39. Maisuradze E, Garishvili T, Bakhutashvili V. Inhibition of Bee Venom Phospolipase A2 activity by Plaferon LB. Bulletin of the Georgian Academy of Sciences 1998; 157, (2): 317-319.
40. Malashhia Y, Beridze M, Bakhutashvili V. Immunomodulator Plaferon Lb in the treatment of acute stroke. Int J Immunorehab 1999; 12: 161-165.
41. Metreveli D, Bakhutashvili V, Bochorishvili T, Jamutashvili M, Gingolava M. Influence of Plaferon-LB on clinical course of Hepatitis and Laboratory Data in Children. International Falk Workshop—New Aspects in Hepatology and Gastroenterology; May 29-30, 1998; Tbilisi, Georgia. Abstract # 195.
42. Metreveli D, Bakhutashvili A, Chikovani T, Pavliashvili D, Bakhutashvili V. Long term results of treatment of B hepatitis with Plaferon LB. Int J Immunorehab 1999; 12: 43. Abstract # 169.
43. Mikeladze D, Djanashia N, Djanashvili C, Bakhutashvili V. Influence of Plaferon upon main excitatory and inhibitary neurotransmitters of brain. Tbilisi 1995; 332-337.
44. Mitagvaria N, Bakhutashvili V, Sanikidze T, Nebieridze M, Pipia N. Plaferon-LB prevents development of cerebral infarction after photochemically induced thrombosis in rats. Georgian J Neurosci 2001; 1: 13-25.
45. Nadareishvili Z, Malashkhia Y, Bakhutashvili V. Plaferon in the treatment of herpes zoster ganglioneuritis in intravenous drug users. Int Conf AIDS, Berlin 1993, June 6-11; 9 (1): 344. Abstract # PO-B08-1254.
46. Nakashidze I, Rukhadze R, Chikovani T, Bakhutashvili V. Influence of Plaferon-LB on structural changes in kidney during experimental traumatic shock. 2001; Proceedings of the Black Sea Countries III International Conference “Advances of Clinical and Theoretical Medicine and Biology”:174-175.
47. Nakashidze I, Rukhadze R, Chikovani T, Bakhutashvili V. Influence of Plaferon-LB on structural changes in myocardium during experimental traumatic shock. 2000; Tbilisi State Medical University, Collection of Scientfic Works Volume XXXIV: 329-332.
48. Nakashidze I, Chikovani T, Gamkrelashvili D, Bakhutashvili V. Oxidation process correction in blood of patients with traumatic shock. Georgian Medical News 2002; 86 (5): 94-98.
49. Pantsulaia I, Chikovani T, Ruhadze R, Sanikidze T, Bakhutashvili V. The impact of Plaferon-LB on changes in immune organs caused by acute experimental hyperthyroidism. Proceedings of the 4th Republic Scientific Practical Conference, Kutaisi, 1998 May 31; Collection of reports: 24.
50. Pantsulaia I, Chikovani T, Cheishvili N, Garishvili T, Kharebava G, Bakhutashvili V, Zhgenti M. Alteration of lymphocytes' proliferative activity in vitro under the influence of plaferon LB fractions. Proc Georgian Acad Sci, Biology Series 1999; 25 (1-6): 75-79.
51. Pantsulaia I, Cheishvili N, Kukuladze N, Jgenti M, Chikovani T. Influence of PlaferonLB on proliferative activity of splenocytes in iexperimental hyper- and hypothyroidism. Int J Immunorehab 2000; 2 (2): 49. Abstract # 157.
52. Pantsulaia I, Pkhakadze E, Cheishvili N, Chikovani T, Jgenti M. Influence of Plaferon LB on the course of moderate periodontitis. Int J Immunorehabilitation 2000; 2 (2): 89. Abstract # 294.
53. Pavliashvili D, Chikovani T, Metreveli D, Sanikidze T, Bakhutashvili V. Influence of sublingual administration of Plaferon-LB on metabolic disorders in viral B hepatitis. Georgian Med News 1999; 9 (54): 65-67.
54. Ruhadze R, Sanikidze T, Bakhutashvili V, Chikovani T, Pantsulaia I, Jgenti M. Influence of plaferon LB on metabolic disorders in liver during experimental hyperthyroidism. Proc Georgian Acad Sci, Biol Ser 1998; 24 (1-6): 333-337.
55. Ruhadze R, Sanikidze T, Bakhutashvili V, Chikovani T, Nikoleishvili L, Pantsulaia I, Jgenti M. An interim report on the effect of Plaferon LB on metabolic changes in myocardium during experimental hyperthyroidism. Proc Georgian Acad Sci, Biol Ser 1998; 24 (1-6): 339-343.
56. Ruhadze R, Sanikidze T, Ciqovani T, Bakhutashvili V. Influence of Plaferon LB on some indices of liver mitochondria during experimental hyperthyroidism. Georgian Medical News 1999; 2: 7-9.
57. Ruhadze R, Chikovani T, Bakhutashvili V, Sanikidze T, Metreveli D, Pantsulaia I, Balarjishvili M. The impact of Plaferon LB on the metabolism of nitric oxide (NO) in thyrotoxicosis. Bulletin of Georgian Academy of Science 1999; 160 (3): 580-582.
58. Ruhadze R, Chikovani T, Pantsulaia I, Bakhutashvili V. The influence of Plaferon LB on several splenic morphometric indices during experimental hyper- and hypothyroidism. Int J Immunorehab 1999; 14: 117. Abstract # 76.
59. Ruhadze R, Chikovani T, Bakhutashvili V, Sanikidze T, Metreveli D, Pantsulaia I, Balarjishvili M. Influence of Plaferon LB on the metabolism of nitric oxide in hypothyroidism. Bulletin of Georgian Academy of Science; 2000, 161 (1): 156-158.
60. Ryazantzeva S, Visotskaja I, Ermilova V, Bakhutashvili V. Immunomorphological changes in breast cancer as a result of preoperative administration of immunomodulator Plaferon. Herald of Oncology National Center, Russian Academy of Science; Clinical Investigations 1999; 4: 37.
61. Shakarishvili R, Sanikidze T, Mitagvaria N, Beridze M, Mikeladze D, Bakhutashvili V. The role of oxygen and nitrogen reactive species in pathogenesis of ischemic stroke. Report on Scientific Session of NATO, Tbilisi, Georgia, October 2001.
62. Sulkhanishvili V, Bakuradze V, Amiridze G, Chigogidze T, Bakhutashvili V. The Effect of Plaferon-LB on local kidney blood flow in acute hemorrhage and septicemia in experiment. Georgian Medical News 2002; 86 (5): 22-24.
63. Tatishvili N, Bakhutashvili V, Simonia G. Local use of Plaferon LB in rheumathology. Int J Immunorehab 1999; 12: 81. Abstract # 303.
64. Telia À, Bakhutashvili V, Jorjoladze N, Kvachadze L. Use of Plaferon LB in children with bronchial asthma. Int J Immunorehab 1997; 7: 139.
65. Telia À, Bakhutashvili V, Kokaia L, Jorjoladze N, Kvachadze L, Alavidze M. Preventive effect of Plaferon LB on hystamine-caused bronchial obstruction in guinea pig. Int J Immunorehab 1998; 9: 28
66. Telia À, Bakhutashvili V, Kokaia L, Alavidze M, Pagava K, Jorjoladze N, Kvachadze L. Plaferon LB as an alternative preparation for treatment of bronchial asthma in children. Int J Immunorehab 1998; 10: 165-167.
67. Telia À, Katsarava V, Bakhutashvili V. Impact of Plaferon LB on bronchial and alveolar cell count and ventilatory function in patients with sthma. Int J Immunorehab 1999; 12 (S): 24.
68. Telia À, Bakhutashvili V, Kokaia L, Alavidze M, Kvachadze L. Comparison of effect of Plaferon LB and chromglycate in adults with bronchial asthma. Int J Immunorehab 1999; 12: 164-165.
69. Olsson T. Critical influences of the cytokine orchestration on the outcome of myelin-antigen specific T cell autoimmunity in experimental auto-immune encephalomyelitis and multiple sclerosis. Immunol. Rev., 1995, 144; 245-268.
70. Lorentzen J C., Issazadeh S., Storch M., Mustafa M I., Lassman H., Linington C., Klareskog L., and Olsson T. Protracted, relapsing and demyelinating experimental auto-immune encephalomyelitis in DA rats immunized with syngeneic spinal cord and incomplete Freund's adjuvant. J. Neuroimmunol., 1995, 63: 193-205.
71. Issazadeh S., Lorentzen J C., Mustafa M I., Hojeberg B., Mussener A., and Olsson T. Cytokines in relapsing experimental auto-immune encephalomyelitis in DA rats: persistent mRNA expression of proinflammatory cytokines and absent expression of interleukin-10 and transfroming growth factor-b. J. Neuroimmunol., 1996, 69: 103-115
72. Bendtzen K. Interleukin-1, interleukin-6 and tumour necrosis factor in infection, inflammation and immunity. Immunol. Lett., 1988, 19: 183-192
73. Rabinovitch A. An update on cytokines in the pathogenesis of insulin-dependent diabetes mellitus. Diab. Metab. Rev., 1998, 14: 129-151
74. Ohlsson K., Bjork P., Bergenfeldt M., Hageman R., and Thompson R C. Interleukin-1 receptor antagonist reduces mortality from septic shock. Nature (Lond), 1990, 348: 550
75. Alexander H R., Doherty G M., Buresh C M., Venzon D J., and Norton J A. A recombinant human receptor antagonist to interleukin-1 improves survival after lethal endotoxaemia in mice. J. Exp. Med., 173: 1029
76. Mattner F., Ozmen L., Podlaski F J., Wilkinson V L., Presky D H., Gately M K., and Alber G. Treatment with homodimeric interleukin-12 (IL-12) p40 protects mice from IL-12-dependent shock but not from tumour necrosis factor alpha-dependent shock. Infection and Immunity, 1997, 65:4734-4737
77. Ashkenazi A., Marsters S A., Capon D J., Chamow S M., Figari I S., Pennica D., Goeddel D V., Palladino M A., and Smith D H. Protection against endotoxin shock by tumor necrosis factor receptor immunoadhesion. Proc. Natl. Acad. Sci. USA, 1991, 88:1035-10539
78. Heinzel F P. The role of IFNgamma in the pathology of experimental endotoxemia. J. Immunol., 1990, 145: 2920-2924
79. Tiegs G., Hentschel J. and Wendel A. A T cell-dependent experimental liver injury in mice inducible by concanavalin A. J. Clin. Invest., 1992, 90: 196-203
80. Gantner F., Leist M., Lose A W., German P G., and Tiegs G. ConcanavalinA-induced T-cell-mediated hepatic injury in mice: The role of tumor necrosis factor. Hepatology, 1995,21:190-198
81. Mizuhara H., O'Neill E., Seki N., Ogawa T., Kusunoki C., Otsuka K., Satoh S., Niwa M., Senoh H., and Fujiwara H. T cell activation-associated hepatic injury: mediation by tumor necrosis factor and protection by interleukin 6. J. Exp. Med., 1994, 179: 1529-1537
82. Holmdahl R. et al., Immunology, 1992, 76: 197-202
83. Svelander L. et al., Immunology, 1997, 91: 260-265
84. Mussener A. Scand. J. Immunol., 1995, 42: 128-134
85. Bach J F. Endocr. Rev., 1994, 15: 516-542
86. Rabinovitch A. Diab. Metab. Rev., 1988, 14: 129-151
87. Rossini A A. et al.,. Clin. Immunol. Immunopathol., 1995, 74: 2-9
88. Parish N M., et al. Immunol. Rev., 1995, 144: 269-300
89. Nicoletti F. et al., Autoimmunity, 1996, 24: 187-197
90. Mori Y. et al, Diabetologia, 1986, 29: 244-247
91. Toyabe S., Seki S., Iaii T., Takeda K., Shirai K., Watanabe H., Hiraide H. et al. Requirements of IL-4 and NK1+T cells for concanavalin A-induced hepatic injury in mice. J. Immunol., 1997, 159: 1537-1542
92. Nicoletti F., Zaccone P., Xiang M., Magro G., Di Mauro M., Di Marco R., Garotta G. et al. Essential pathogenetic role for interferon (IFN)-gamma in Concanavalin A-induced T cell-dependent hepatitis: exacerbation by exogenous IFN-g and prevention by IFN-g receptor-immunoglobulin fusion protein. Cytokine 2000, 12: 315-323
93. Di Marco R., Xiang M., Zaccone P., Leonardi C., Franco S., Meroni P., Nicoletti F. Concanavalin A-induced hepatitis in mice is prevented by interleukin (IL)-10 and exacerbated by endogenous IL-10 deficiency. Autoimmunity, 1999, 31: 75-83
94. Nicoletti F., Di Marco R., Zaccone P., Salvaggio A., Magro G., Bendtzen K., and Meroni P L. Murine Concanavalin A-induced hepatitis is prevented by interleukin-12 (IL-12) antibody and exacerbated by exogenous IL-12 through an interferon-gamma-dependent mechanism. Hepatology, 2000, 32: 728-733
95. Dzotsenidze, L., Mkervalishvili, J., Gogiashvili, R., Bakhutashvili, V. The Antitoxic Effect of Plaferon LB on Pesticides in Tissue Culture Model. Annals of Biomedical Research and Education, 2003, Vol. 3, Issue 3, p. 180-182.
96. Chichua, G., Sanikidze, T., Bakhutashvili, V. Biochemical Changes in Retinal Tissue During Vitreoretinal Pathology. Annals of Biomedical Research and Education, 2002, Vol. 2, Issue 3, p. 189-195.
97. Kvezereli, M., Chikovani, T., Sanikidze, T., Rukhadze, R., Cheishvili, N., Sharashenidze, T., Papava, M., Bakhutashvili, V. Effects of Plaferon LB (PLB) on Concanavalin A-induced hepatitis in Mice. Annals of Biomedical Research and Education, 2002, Vol. 2, Issue 2, p. 133-138.
98. Bakradze, I., Bakhutashvili, V., Aladashvili, A., Shavdia, M., Aroshidze, T. The Perioperative Immunocorrection by Plaferon for Colorectal Cancer Patients. Annals of Biomedical Research and Education, 2002, Vol. 2, Issue 1, p. 38-41.
99. Maisuradze, M. Abdushelishvili, N., Dumbadze, M. Some Aspects of Protective Effect of Plaferon LB During Bronchial Asthma. Annals of Biomedical Research and Education, 2003, Vol. 3, Issue 3, p. 189-191.
100. Lobzhanidze, N., Koupradze, S. Positive Effect of Plaferon-LB on Treatment of Hodgkin's Disease—Result of Immunocorrection or Apoptosis? Annals of Biomedical Research and Education, 2001, Vol. 1, Issue 3, p. 170-173.
101. Mann, M. and Jensen, O. N. Proteomic Analysis of Post-Translational Modifications. Nature Biotechnology, 2003, Vol. 21, p. 255-261.
102. Jensen, L. J., Gupta, R., Blom, N. Devos, D., Tamames, J., Kesmir, C., Nielsen, H., Staerfeldt, H. H., Rapacki, K., Workman, C., Andersen C. A. F., Knudsen, S., Krogh, A., Valencia, A. and Brunak S. Prediction of Human Protein Funcation from Post-Translational Modifications and Localization Features. J. Mol. Biol., 2002, 319: 1257-1265.
103. Proteomics: Post Translational Modification, Pierce Biotechnology, Inc. web site, accessed Nov. 11, 2004, <http://www.piercenet.com/Proteomics/browse.cfm?fldID=7CE3F CF5-0DA0-4378-A513-2E35E5E3B49B>.
104. Topical Preparations, PsoriasisNet, American Academy of Dermatology, accessed Nov. 11, 2004, <http://www.skincarephysicians.com/psoriasisnet/TopicalPrepa rations.html>.
105. Djavachishvili N., Bachutashvili, W., Tsagareli Z. Die wirkung von Plaferon heim experimentellen myokardinfarkt. Verh Anat Ges 1991, 84: 301-2 (in German).
106. Gibradze, T., Bakhutashvili, V., Lortkipanidze, T. Influence of Plaferon on myocardium and its blood vessels in injury of heart by adrenaline. Kardiologia 1994, 3 :42-3. (in German)
107. Kacarava, Z. Effect of Plaferon on development of experimental brain focal ischemia induced by photochemical thrombosis. Tbilisi 1995, p. 23.
108. Chavchanidze, D. Protective effect of Plaferon in obstructive nephropathy. Tbilisi 1990, p. 8-14.
109. Xvadagiani, G. Protective effect of Plaferon in ischemia of kidney. Tbilisi 1990, 1-22.
110. Beridze, M. Bakhutashvili, V. Impact of Plaferon-LB upon clinical and immunity indices in patients with ischemic stroke. Georgian Medical News 1998, 39: 27-30.
111. Beridze, M. Bakhutashvili, V., Malashkhia, I. Plaferon-LB in therapy of patients with ischemic stroke. Int. J. Immunorehab 1999, 12: 27 and 161.
112. Dumbadze M., Sanikidze, T., Gongadze, M., Bakhutashvili, V. Plaferon LB modulates NOS activity in different pathologies. Int. J. Immunorehab, 2002, vol. 4, 1: 72.
113. Lezhava, G., Todadze, K. H., Mikeladze, D., Sanikidze, T. Influence of Metadoxil, Plaferon-LB and P-6 on the amino acid content, nitrogen oxide synthesis and intensity of the EPR signals int he brain of alcoholized rats. Innovative Strategien in der Therapie psychiatrischer Erkrankungen. Potsdam, 2000, p. 54.
114. Ruhadze R., Sanikidze, T., Chiqovani, T., Bakhutashvili, V. Impact of Plaferon LB on structural and functional changes in mitochondria of hepatocites under the conditions of experimental hyperthyroidosis. Georgian Medical News, 1999, 2: 3-5.
115. Nanava G. I., Sanikidze T. V., Chikovani T. I., Bakhutashvili V. I., Shengelaya N. V., Nanava V. I. “Plaferon-LB influence on Electron Spin Resonance indices of blood in acute period after surgery for brain tumor.” Georgian Medical News 2000, 9 (66).
116. Chavchanidze D., Managadze L., Bakhutashvili V., Sanikidze T. “Determination of traumatic influence of shock waves and membrane protector effects of agent Plaferon-LB on renal parenchyma during extracorporal lithotripsy in experiment.” Exper Biol and Med 1998 vol 24, (1-3) pp 65-72.
117. Gabisiani Kh., Bakhutashvili V., Sanikidze T., Mitagvaria N., Devdariani M., Pipia N. Plaferon-LB as a modulator of reactive forms of nitrogen and oxygen during photochemical-induced stroke. Proc Georgian Acad Sci, Biol. Ser., 2000, vol. 26, No 1-3.
118. Javakhisvili N., Tsagareli Z., Sanikidze T., Machavariani M., Bakhutashvili Z., Enukidze M., Kharebava G., v. Bakhutashvili V. Antioxidative effect of Plaferon and Plaferon LB in experimental IM. Experimental and clinical medicine, 2001, 2, p. 11-15.
119. Nakashidze I., Chikovani T., Sanikidze T., Jgenti M., Bakhutashvili V. The dynamics of lipid peroxidation and condition of antioxidant system in traumatic shock. Int J Immunorehab, 2002 v4, 1, p140
120. Shakarashvili R., Sanikidze T., Mitagvaria N., Beridze M., Mikeladze D., Bakhutashvili V. The Role of oxygen and nitrogen reactive species in pathogenesis of ischemic stroke. NATO Science Series. D. Horeman., v. 342, pp. 171-182. IOSPRESS, Netherlands.
121. Gongadze M, Chikovani T., Sanikidze T,m Bakhtashvili V. Correction of LPS-induced Nitric Oxide Synthesis. Bulletin of Georgian Academy of Sciences. 2002, 311-314.
122. Sharashenidze T., Sanikidze T., Chikovani T., Cheishvili N., Bakhutashvili V. Pro- and anti-oxidative systems in Con A induced hepatitis. Georgian Medical News 2001 12(81) 108-111.

1. An isolated peptide comprising an amino acid sequence selected from the group consisting of SEQ ID NOs. 1-5 or functional equivalent thereof.

2. (canceled)

3. An isolated nucleic acid molecule encoding a peptide comprising an amino acid sequence selected from the group consisting of SEQ ID NOs. 1-5 or a functional equivalent thereof.

4. A vector comprising the nucleic acid molecule of claim 3.

5. A cell comprising the vector of claim 4.

6-10. (canceled)

11. A composition comprising the isolated peptide of claim 1 and a suitable carrier.

12-13. (canceled)

14. The composition according to claim 11, wherein the carrier is suitable for topical, sublingual, parenteral or gastrointestinal administration or aerosolization.

15. (canceled)

16. A method for improving the skin condition of a subject comprising contacting an effective amount of the peptide of claim 1 with a skin surface on the subject.

17-34. (canceled)

35. The method of claim 16, wherein said skin surface comprises a wound and contacting with said peptide facilitates wound healing.

36-50. (canceled)

51. A method for protecting against the effects of Tumor Necrosis Factor (TNF) in a cell comprising contacting said cell with an effective amount of the composition of claim 11.

52-59. (canceled)

60. A method for treating septic shock or Gram negative sepsis in a subject comprising administering to the subject an effective amount of the composition of claim 11.

61-89. (canceled)

90. A method of improving the skin condition of a subject comprising contacting an effective amount of a tripeptide NVS or NVSp with a skin surface on the subject.

91-210. (canceled)

211. An antibody that binds to the peptide of claim 1.

212. A method of regulating T cell function in a subject comprising administering to the subject an effective amount of the composition of claim 11.


SEQUENCE LISTING (5 sequences)

SEQ ID NO: 1 - protein, 8 aa, Homosapien
NVSAVEIA

SEQ ID NO: 2 - protein, 8 aa, Homosapien
  MISC_FEATURE (3)..(3): Phosphorylated at this location.
NVSAVEIA

SEQ ID NO: 3 - protein, 8 aa, Homosapien
NVAAVEIA

SEQ ID NO: 4 - protein, 8 aa, Homosapien
NVCKVEIA

SEQ ID NO: 5 - protein, 8 aa, Homosapien
NVFKVEIA